(19) United States
(12) Patent Application Publication
(10) Pub. No.: US 2010/0255481 A1
(43) Pub. Date: Oct. 7, 2010

(54) METHOD FOR DETECTION OF ADENOMA OR CANCER BY GENETIC ANALYSIS

(75) Inventors: Tamao Akesaka, Tokorozawa-shi (JP); Tomonori Nagaoka, Tokyo (JP); Shigeru Kanaoka, Hamamatsu-shi (JP); Kenichi Yoshida, Hamamatsu-shi (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignees: OLYMPUS CORPORATION, Tokyo (JP); NATIONAL UNIVERSITY CORPORATION HAMAMATSU; UNIVERSITY SCHOOL OF MEDICINE, SHIZUOKA (JP)

(21) Appl. No.: 12/740,283

(22) PCT Filed: Oct. 30, 2008
(86) PCT No.: PCT/JP2008/069757

§ 371 Date: Apr. 28, 2010

(30) Foreign Application Priority Data
Oct. 30, 2007 (JP) 2007-282044
Aug. 29, 2008 (JP) 2008-222748
Sep. 30, 2008 (JP) 2008-252649

(51) Int. Cl. C12Q 1/68

(52) U.S. Cl. 435/6

(57) ABSTRACT

Disclosed is a method which enables an early stage detection of an adenoma or cancer by gene expression analysis of a biomarker in a readily collectable sample. Specifically disclosed is a method for detecting an adenoma or cancer, which comprises the steps of: measuring the quantity of a sequence constituting at least one housekeeping gene or an expression product thereof contained in a body fluid sample or an excrement sample collected from an examinee; and calculating the concentration of the sequence in the sample.

### TECHNICAL FIELD

The present invention relates to a method for detecting an adenoma or cancer by genetic analysis. More specifically, the present invention relates to a method which enables an early stage detection of an adenoma or cancer by genetic analysis of a biomarker in a readily collectable sample.

### BACKGROUND ART

Colorectal cancer is the top leading cause of death in Japan and the second leading cause of cancer death in the United States. In the United States, about 1,300,000 cases of colorectal cancer are found each year, and of these about 50,000 people die, making it the third leading cause of death. Therefore, measures to counter cancer must be urgently adopted.

In most cases, colorectal cancer is started from a small benign adenoma and is slowly developed into a malignant tumor over several tens of years. Thus, if it is found at an early stage, surgical treatments are effective and complete recovery is possible.

In the case of a benign adenoma, a low invasive endoscopic resection can be done. Even in the case of a malignant tumor, if it is at an early stage, an endoscopic resection can be done. Furthermore, even in the case of advanced cancer, surgical treatments are often effective. Because of such a slow development process, there are many chances to prevent and intervene this disease. Accordingly, it is possible to reduce the morbidity rate and the mortality rate of colorectal adenoma or tumor by early stage detection and resection.

However, currently performed adenoma or cancer detection methods, such as screening test methods for colorectal adenoma or tumor (including a fecal occult blood test, double contrast barium enema, sigmoidoscopy, and total colonoscopy) involve various problems.

The fecal occult blood test is to detect a bleeding adenoma or tumor indirectly by checking blood contained in feces. However, many cases of early stage adenoma or tumor may result in false negatives, and thus the sensitivity can not be said to be sufficient. Moreover, cases of bleeding which occurs not from an adenoma or tumor but from an intestinal tract (such as hemorrhoid) may result in false positives, and thus the specificity can not be said to be high.

The barium enema is an X-ray photographic method in which barium and air are injected from the anus after a thorough laxative pretreatment. This test method can clarify the accurate position and size of cancer, the degree of narrowness of the intestine, and the like. Therefore, it is possible to detect a large-shaped advanced cancer. However, the shortcoming is that it is difficult to detect a small-shaped early stage cancer or a flattened cancer.

The sigmoidoscopy and the total colonoscopy are videoscopic methods in which the inside of the intestine is observed after a thorough laxative pretreatment. The laxative pretreatment in these test methods requires the administration of two to three liters of laxative, which imposes an unpleasant burden on the examinee. Furthermore, tearing or perforation may occur during the test. For this reason, these methods are regarded as not appropriate for the screening test.

In this manner, the current test methods as mentioned above can not be said to satisfy the necessary and sufficient performance for checking an adenoma or cancer. Therefore, there is a demand for a low invasive test method which has high sensitivity and high specificity.

Patent Document 1 and Non-patent Document 1 disclose methods for testing colon cancer based on the difference in the fragment length of the Alu repeat region, the alphoid repeat region, the p53, or such a cancer-related gene so as to detect non-apoptotic DNA. In these methods, DNA has to be extracted, although it is difficult to recover fragmented DNA and it is also difficult to amplify fragmented DNA. Therefore, these methods are problematically inferior in the detection sensitivity in the end. In addition, even if a fragment can be detected, there is a fundamental problem in that it is not possible to discriminate the origin of the fragment, that is, whether the fragment is derived from cancer, or caused by some damage during the procedure. Therefore, there is a need of a new marker which can indicate the presence of colon cancer by a different means other than DNA fragmentation.

Patent Document 2 relates to a method for quantifying a gene, which comprises: performing PCR with the presence of an internal standard gene; subjecting a gene which serves as a detection target and the internal standard gene to a PCR reaction; independently measuring the thus amplified genes resulting from these genes by enzyme immunoassay; and using the signal ratio between these two kinds of amplified genes. Since the correction is performed with use of the internal standard gene, the gene as the detection target can be more quantitatively and more accurately examined. However, since the internal standard gene is individually added to all test samples, it is difficult to correct the difference between prepared samples per se although it is possible to correct the difference between experiments caused by PCR reactions. For this reason, the problem is that, when using a body fluid sample or an excrement sample such as feces which largely differs per each sample, it is not always easy to obtain a highly reliable result.

On the other hand, because feces is used as a specimen, the test can be done less invasively and the burden on the examinee for the test can be greatly alleviated. Therefore, if there is a method which can detect an adenoma or cancer by means of nucleic acid analysis in feces, the method can be expected to prevail as a screening test substituting for the fecal occult blood test.

However, the use of feces as a specimen involves several problems because of the nature in itself. First, generally, various types of substances are mixedly present in feces, and substances derived from cancer cells or pathogenic bacteria account for a very small proportion. That is, the problem is that the detection sensitivity and accuracy may be sometimes insufficient because the quantity of nucleic acids of human-derived cells is relatively small with respect to the quantity of feces, and the extraction of these nucleic acids is also difficult. In addition, many nucleases and such enzymes are contained in impurities within feces, and thus another problem is that the quality and the quantity of nucleic acids of human-derived cells in feces are easily changeable depending on the time elapsed from the time of collection and the surrounding environment such as the temperature.

In order to solve these problems, various methods are disclosed. For example, there is a method (1) for diagnosing a patient for the presence of colon cancer, which comprises the steps of: (a) measuring levels of a plurality of CSGs (colon specific genes) in a cell, tissue, or body fluid sample collected from the patient; (b) comparing the measured CSG levels with CSG levels of a control sample; and determining that the patient is a candidate for colon cancer if the CSG levels of the patient are above the CSG levels of the control sample (for example, refer to Patent Document 3). This method improves the detection sensitivity for colon cancer by increasing the number of CSGs to be measured, that is, the number of markers. In addition, there is also disclosed a method (2) in a form of a test flowchart, which comprises the steps of: quantifying a sample nucleic acid to determine whether or not the result is above a threshold; and then either carrying on the test if the result is above the threshold, or terminating the test if the result is below the threshold by determining that the examinee who provided the sample is healthy (for example, refer to Patent Document 4).

### Patent Document 1: Published Japanese Translation No. 2005-514073 of the PCT International Publication

Patent Document 2: Japanese Unexamined Patent Application, First Publication No. H07-303499

### Patent Document 3: Published Japanese Translation No. 2002-515591 of the PCT International Publication

### Patent Document 4: United States Patent Application, Publication No. 2004/0259101

Non-patent Document 1: Boynton et al. “DNA Integrity as a Potential Marker for Stool-based Detection of Colorectal Cancer”, Clinical Chemistry Vol. 49, No. 7, p. 1058-1065, 2003

### DISCLOSURE OF INVENTION

Prior art methods for detecting an adenoma or cancer involve these problems mentioned above. In order to solve such problems, the present invention aims to provide a method which can enable an early stage detection of an adenoma or cancer by genetic analysis of a biomarker in a readily collectable sample.

In particular, the above-mentioned method (1) compares only the quantitative results as a comprehensive evaluation of quantitative values of a plurality of markers, and does not consider the validity of the process for measuring the samples. That is, no specific control is given to each measurement process, and a means for understanding whether or not the measurement process is successful is out of consideration. For this reason, there is left a problem in that false negatives and false positives may occur if any error happens during the measurement process. Moreover, the target of this method is primarily a body fluid such as blood, and there is no consideration at all regarding the reliability of the test when using a sample such as feces which contains many impurities.

On the other hand, the above-mentioned method (2) has only the quantitative criteria for the nucleic acid sample and does not consider any qualitative criteria. For this reason, there is left a problem in that false negatives and false positives may occur in the case of any qualitative issue such as a bad quality of the nucleic acid (decomposed or fragmented) and a bad quality of a reagent.

It is an object of the present invention to provide a method which enables an early stage detection of an adenoma or cancer by genetic analysis of a biomarker, particularly in a fecal sample, and which can provide highly reliable results.

In order to solve such problems and to achieve the above object, the present invention takes the following structures.

(1) An adenoma or cancer detection method which comprises the steps of: measuring the quantity of a sequence constituting at least one housekeeping gene or an expression product thereof contained in a body fluid sample or an excrement sample that has been collected from an examinee; and calculating the concentration of the sequence in the sample.

(2) An adenoma or cancer detection method which comprises: (i) a step of extracting a nucleic acid or a protein from a body fluid sample or an excrement sample that has been collected from an examinee; and (ii) a step of measuring the quantity of a sequence constituting at least one housekeeping gene or an expression product thereof in the extracted nucleic acid or protein, and calculating the concentration of the sequence in the sample.

(3) The adenoma or cancer detection method according to (2), wherein the method further comprises: (a) a step of homogenizing the sample, and appropriately freezing or freeze-drying it at 0° C. or lower temperature, or treating it with alcohol or an alcoholic solution, before the step (i).

(4) The adenoma or cancer detection method according to (2), wherein the method further comprises: (b) a step of producing a cDNA from an RNA which is derived from at least one housekeeping gene among the RNA extracted in the step (i), between the step (i) and the step (ii); and quantifying the cDNA in the step (ii).

(5) The adenoma or cancer detection method according to either one of (1) and (2), wherein the body fluid sample is any one of saliva, sputum, nasal mucus, lacrimal fluid, gastric juice, bile, pancreatic juice, sweat, cerebrospinal fluid, pus, pleural effusion, cardiac effusion, milk, vaginal secretion, semen, ascites, amniotic fluid, lymph fluid, and blood, and the excrement sample is feces or urine.

(6) The adenoma or cancer detection method according to either one of (1) and (2), wherein there are two or more types of the housekeeping genes, and a plurality of quantities of sequences constituting these housekeeping genes or expression products thereof are measured at the same time.

(7) The adenoma or cancer detection method according to (6), wherein the housekeeping gene is a gene selected from the group consisting of glyceraldehyde-3-phosphate dehydrogenase (GAPDH), 18S ribosomal RNA, 28S ribosomal RNA, (β actin, (β2 microglobulin, hypoxanthine phosphoribosyl transferase 1, ribosomal protein large P0, peptidylpropyl isomerase A (cyclosporin A), cytochrome C, phosphoglycerate kinase 1, β-glucuronidase, TATA box binding factor, transferrin receptor, HLA-A0201 heavy chain, ribosomal protein L19, α tubulin, β tubulin, γ tubulin, ATP synthetase, eukaryotic translation elongation factor 1 gamma (EEF1G), succinate dehydrogenase complex (SDHA), aminolevulinic acid synthase 1 (ALAS1), ADP-ribosylation factor 6, endonuclease G (ENDOG), and peroxisomal biogenesis factor (PEX).

(8) The adenoma or cancer detection method according to either one of (1) and (2), wherein the excrement sample is feces and the housekeeping gene is β2 microglobulin.

(9) The adenoma or cancer detection method according to (2) wherein the method further comprises, after the step (ii): (iii) a step of measuring the quantity of a sequence constituting at least one tumor gene or an expression product thereof in the extracted nucleic acid or protein, and calculating the concentration of the sequence in the sample; and (iv) a step of correcting the concentration of the sequence constituting the tumor gene or the expression product thereof that has been calculated in the step (iii), based on the concentration of the sequence constituting the housekeeping gene or the expression product thereof that has been calculated in the step (ii).

(10) An adenoma or cancer testing method for testing an adenoma or cancer with use of a marker gene for the adenoma or cancer (hereinunder, referred to as the target gene), which comprises the following steps (provided that the steps (B), (C), (D), and (E) may be carried out in the order of the steps (D), (E), (B), and (C), the steps (B), (D), (C), and (E), the steps (B), (D), (E), and (C), the steps (D), (B), (E), and (C), or the steps (D), (B), (C), and (E)):

(A) a step of extracting an RNA contained in feces that has been collected from an examinee, and purifying it as an RNA solution;

(B) a step of measuring the quantity of a target gene-derived RNA in the RNA solution obtained in the step (A);

(C) a step of comparing the quantity of the target gene-derived RNA obtained in the step (B) with a preset threshold, to determine whether or not the examinee is affected by the adenoma or cancer;

(D) a step of measuring one or more items selected from the group consisting of the degree of RNA purification, the degree of RNA decomposition, the RNA concentration, and the quantity of a standard gene-derived RNA, in the RNA solution obtained in the step (A);

(E) a step of judging the reliability of the RNA in the RNA solution obtained in the step (A), based on the value obtained in the step (D); and

(F) a step of judging that the determination of the step (C) is reliable if the RNA is judged to be reliable in the step (E), and judging that the determination of the step (C) is unreliable if the RNA is judged to be unreliable in the step (E).

(11) The adenoma or cancer testing method according to (10), wherein the steps (B), (C), (D), and (E) are carried out in the order of the steps (D), (E), (B), and (C), the step (B) is the following step (B1), and the step (C) is the following step (C1):

(B1) a step of terminating the test if the RNA is judged to be unreliable in the step (E), or measuring the quantity of a target gene-derived RNA in the RNA solution obtained in the step (A) if the RNA is judged to be reliable in the step (E); and

(C1) a step of comparing the quantity of the target gene-derived RNA obtained in the step (B1) with a preset threshold, to determine whether or not the examinee is affected by the adenoma or cancer.

(12) The adenoma or cancer testing method according to (10), wherein the steps (B), (C), (D), and (E) are carried out in the order of the steps (B), (D), (E), and (C), the step (D) is the following step (D2), the step (E) is the following step (E2), and the step (C) is the following step (C2):

(D2) a step of measuring one or more items selected from the group consisting of the degree of RNA purification, the degree of RNA decomposition, the RNA concentration, and the quantity of a standard gene-derived RNA, in the RNA solution obtained in the step (A), after the step (B);

(E2) a step of judging the reliability of the RNA in the RNA solution obtained in the step (A), based on the value obtained in the step (D2); and

(C2) a step of terminating the test if the RNA is judged to be unreliable in the step (E2), or comparing the quantity of the target gene-derived RNA obtained in the step (B) with a preset threshold, to determine whether or not the examinee is affected by the adenoma or cancer, if the RNA is judged to be reliable in the step (E2).

(13) An adenoma or cancer testing method for testing an adenoma or cancer with use of a marker gene for the adenoma or cancer (hereinunder, referred to as the target gene), which comprises the following steps:

(A) a step of extracting an RNA contained in feces that has been collected from an examinee, and purifying it as an RNA solution;

(B′) a step of measuring the quantity of a target gene-derived RNA and the quantity of a standard gene-derived RNA in the RNA solution obtained in the step (A);

(C′) a step of determining that the examinee is affected by the adenoma or cancer if a value resulting from the division of the quantity of the target gene-derived RNA obtained in the step (B′) by the quantity of the standard gene-derived RNA obtained in the step (B′) is greater than a preset threshold, and determining that the examinee is unaffected by the adenoma or cancer if the above-mentioned value is smaller than the preset threshold;

(D) a step of measuring one or more items selected from the group consisting of the degree of RNA purification, the degree of RNA decomposition, the RNA concentration, and the quantity of the standard gene-derived RNA, in the RNA solution obtained in the step (A);

(E) a step of judging the reliability of the RNA in the RNA solution obtained in the step (A), based on the value obtained in the step (D); and

(G′) a step of judging that the determination of the step (C′) is reliable if the RNA is judged to be reliable in the step (E), and judging that the determination of the step (C′) is unreliable if the RNA is judged to be unreliable in the step (E).

(14) The adenoma or cancer testing method according to any one of (10) to (13), wherein the measurement of the degree of RNA purification is carried out by measuring a value resulting from the division of the absorbance at 260 nm by the absorbance at 230 nm (260/230 nm absorbance ratio) of the RNA solution obtained in the step (A), and/or a value resulting from the division of the absorbance at 260 nm by the absorbance at 280 nm (260/280 nm absorbance ratio) thereof.

(15) The adenoma or cancer testing method according to (14), wherein the RNA obtained in the step (A) is judged to be unreliable if the 260/230 nm absorbance ratio or the 260/280 nm absorbance ratio is smaller than 1.0 or greater than 2.5.

(16) The adenoma or cancer testing method according to any one of (10) to (15), wherein the measurement of the degree of RNA decomposition is carried out by measuring a value resulting from the division of the quantity of a fragment of 23 S ribosomal RNA by the quantity of a fragment of 16S ribosomal RNA (23S rRNA/16S rRNA ratio) of the RNA in the RNA solution obtained in the step (A).

(17) The adenoma or cancer testing method according to (16), wherein the RNA obtained in the step (A) is judged to be unreliable if the 23S rRNA/16S rRNA ratio is smaller than 1.6 or greater than 2.5.

(18) The adenoma or cancer testing method according to any one of (10) to (17), wherein the RNA obtained in the step (A) is judged to be unreliable if the RNA concentration in the RNA solution obtained in the step (A) is lower than 10 ng/μL.

(19) The adenoma or cancer testing method according to any one of (10) to (18), wherein the RNA obtained in the step (A) is judged to be unreliable if the quantity of the standard gene-derived RNA is smaller than a preset threshold.

(20) The adenoma or cancer testing method according to any one of (10) to (19), wherein the measurement of the quantity of the target gene-derived RNA is carried out after normalization of the RNA solution obtained in the step (A).

(21) The adenoma or cancer testing method according to any one of (10) to (19), wherein the measurement of the quantity of the target gene-derived RNA is carried out by nucleic acid amplification with use of a cDNA, as a template, which has been obtained from a reverse transcription reaction of the RNA in the RNA solution obtained in the step (A).

(22) The adenoma or cancer testing method according to any one of (10) to (19), wherein the measurement of the quantity of the target gene-derived RNA is carried out by performing a reverse transcription reaction after normalization of the RNA solution obtained in the step (A), and nucleic acid amplification with use of a resulting cDNA as a template.

(23) The adenoma or cancer testing method according to (13), wherein the measurement of the quantity of the target gene-derived RNA and the quantity of the standard gene-derived RNA in the step (B′) is carried out by the multiplex PCR.

(24) The adenoma or cancer testing method according to any one of (10) to (23), wherein the standard gene is a housekeeping gene or an epithelial cell-specific gene.

(25) The adenoma or cancer testing method according to (24), wherein the epithelial cell-specific gene is a gene selected from the group consisting of a carcinoembryonic antigen gene, a cell adhesion factor gene, a mucin gene, and a cytokeratin gene.

(26) The adenoma or cancer testing method according to any one of (10) to (25), wherein the target gene is a gene selected from the group consisting of cyclooxygenase 2 (COX2), matrix metallopeptidase 7 (MMP7), and SNAIL.

(27) The adenoma or cancer testing method according to (24), wherein the standard gene is a gene selected from the group consisting of glyceraldehyde-3-phosphate dehydrogenase (GAPDH), 18S ribosomal RNA, 28S ribosomal RNA, β actin, β2 microglobulin, hypoxanthine phosphoribosyl transferase 1, ribosomal protein large P0, peptidylpropyl isomerase A (cyclosporin A), cytochrome C, phosphoglycerate kinase 1, β-glucuronidase, TATA box binding factor, transferrin receptor, HLA-A0201 heavy chain, ribosomal protein L19, α tubulin, β tubulin, γ tubulin, ATP synthetase, eukaryotic translation elongation factor 1 gamma (EEF1G), succinate dehydrogenase complex (SDHA), aminolevulinic acid synthase 1 (ALAS1), ADP-ribosylation factor 6, endonuclease G (ENDOG), peroxisomal biogenesis factor (PEX), carcinoembryonic antigen (CEA), epithelial cell adhesion molecule (EpCAM), mutin 2 (MUC2), mutin 3 (MUC3), mutin 4 (MUC4), keratin 7 (CK7), keratin 19 (CK19), and keratin 20 (CK20).

In the adenoma or cancer detection method of the present invention, the detection target is not a fragmented gene that involves various problems in the detection, but instead a housekeeping gene or its expression product which resides in both normal cells and adenoma or cancer-derived cells (more abundantly than a cancer specific gene) is extracted from a sample which is suspected to specifically include living adenoma or cancer-derived cells, and quantified. Therefore, as compared to prior art detections for a cancer specific gene and the like, more sensitive and simpler cancer detection can be achieved.

In addition, the adenoma or cancer testing method of the present invention can enable to check the quality and the quantity of a nucleic acid specimen (RNA, cDNA, or an amplified product) that has been extracted/purified from a biological sample, as well as checking the reagents used, the manipulation, and the process, at stages during the test. In this manner, false negatives and false positives can be avoided by checking during the test regarding whether or not the nucleic acid specimen is in a good state and whether or not the test status is successful, and therefore more reliable test results can be given. Moreover, it becomes possible to specify the inappropriate point in the test flow by performing the test while checking the state of the nucleic acid specimen. As a result, it becomes readily possible to return to the inappropriate point in the test flow, and thereby wasteful time and cost for specifying the point can be reduced.

### BRIEF DESCRIPTION OF THE DRAWINGS

FIG. 1 shows the results of a real-time PCR assay of a normal sample (sample with a cancer cell line) and cancer samples (samples without a cancer cell line) in Example 1.

FIG. 2 shows the results of a real-time PCR assay of a healthy subject sample and colon cancer samples in Example 2.

FIG. 3 shows the results of a real-time PCR assay of healthy subject samples, an adenoma sample, and colon cancer samples in Example 3.

FIG. 4 shows the results of a multiplex real-time PCR assay of a normal sample (sample with a cancer cell line) and colon cancer samples (samples without a cancer cell line) in Example 5.

FIG. 5 shows the results of a real-time PCR assay of a normal sample (healthy subject sample) and cancer samples in Example 6.

FIG. 6 shows the respective B2M gene copy numbers of a control group and a colon cancer patient group in Example 9.

FIG. 7 shows the respective B2M gene copy numbers of the control group and colon cancer patient groups at respective stages in Example 9.

FIG. 8 shows a flowchart of an embodiment of the adenoma or cancer testing method of the present invention.

FIG. 9 shows a flowchart of another embodiment of the adenoma or cancer testing method of the present invention.

FIG. 10 shows a flowchart of yet another embodiment of the adenoma or cancer testing method of the present invention.

FIG. 11 shows signals from nucleic acid amplification on respective samples in the detection of COX2 gene-derived nucleic acid in Example 11.

FIG. 12 shows signals from nucleic acid amplification on respective samples in the detection of GAPDH gene-derived nucleic acid in Example 11.

FIG. 13 shows signals from nucleic acid amplification of the COX2 gene-derived nucleic acids on respective samples when the detection of COX2 gene-derived nucleic acid and GAPDH gene-derived nucleic acid was performed by the multiplex PCR in Example 11.

FIG. 14 shows signals from nucleic acid amplification on respective samples in the detection of IGF-1 gene-derived nucleic acid in Example 12.

FIG. 15 shows signals from nucleic acid amplification on respective samples in the detection of B2M gene-derived nucleic acid in Example 12.

FIG. 16 shows the ratios of IGF-1 expression level/B2M expression level (value resulting from the division of the quantity of IGF-1 gene-derived nucleic acid by the quantity of B2M gene-derived nucleic acid) of respective samples when the detection of IGF-1 gene-derived nucleic acid and B2M gene-derived nucleic acid was performed by the multiplex PCR in Example 12.

### BEST MODE FOR CARRYING OUT THE INVENTION

The adenoma or cancer detection method of the present invention includes the steps of: measuring the quantity of a sequence constituting at least one housekeeping gene or an expression product thereof contained in a body fluid sample or an excrement sample that has been collected from an examinee; and calculating the concentration of the sequence in the sample. Here, the term “housekeeping gene” refers to a gene that is expressed at a substantially constant level at all times in almost all kinds of cells throughout species and creatures, being involved in basic functions needed for cells to survive (such as protein synthesis required for maintenance and proliferation of cells).

Examples of the housekeeping gene can include glyceraldehyde-3-phosphate dehydrogenase (GAPDH), 18S ribosomal RNA, 28S ribosomal RNA, β actin, β2 microglobulin, hypoxanthine phosphoribosyl transferase 1, ribosomal protein large P0, peptidylpropyl isomerase A (cyclosporin A), cytochrome C, phosphoglycerate kinase 1, β-glucuronidase, TATA box binding factor, transferrin receptor, HLA-A0201 heavy chain, ribosomal protein L19, α tubulin, β tubulin, γ tubulin, ATP synthetase, eukaryotic translation elongation factor 1 gamma (EEF1G), succinate dehydrogenase complex (SDHA), aminolevulinic acid synthase 1 (ALAS1), ADP-ribosylation factor 6, endonuclease G (ENDOG), and peroxisomal biogenesis factor (PEX). However, the housekeeping gene for use in the present invention is not limited to these genes.

In the adenoma or cancer detection method of the present invention, the quantity of a sequence constituting a housekeeping gene, that is, the quantity of a DNA-constituting nucleotide sequence, can be measured. Alternatively, instead of the nucleotide sequence, it is also possible to measure the quantity of a nucleotide sequence of mRNA, or the quantity of an amino acid sequence of a protein, which are expression products of the housekeeping gene.

Next, the thus measured quantity of the gene or its expression product is divided by the volume of the sample used for the measurement, to thereby obtain the concentration of the gene or its expression product in the sample. This process enables the quantitative comparison among detection results of a plurality of samples.

More specifically, the adenoma or cancer detection method of the present invention can include the following steps:

(i) a step of extracting a nucleic acid or a protein from a body fluid sample or an excrement sample that has been collected from an examinee; and

(ii) a step of measuring the quantity of a sequence constituting at least one housekeeping gene or an expression product thereof in the extracted nucleic acid or protein, and calculating the concentration of the sequence in the sample.

In the present invention, a nucleic acid or a protein is detected, for which, firstly in the step (i), the nucleic acid or the protein is subjected to an extraction treatment. Since a body fluid sample or an excrement sample is used for this invention, the sample contains many impurities besides the housekeeping gene or its expression product of interest. Accordingly, in order to efficiently detect the housekeeping gene or its expression product, the sample is desirably subjected to a preliminary purification step. The sample collected from an examinee is suspended in a solution such as PBS, and cells are dispersed in the solution by using a homogenizer or such a device. Then, this solution is centrifuged to thereby separate large density impurities as a precipitate and a cell-containing fraction as a supernatant.

This supernatant is subjected to nucleic acid purification or protein purification. As to the method of nucleic acid purification or protein purification, a known method in the art can be employed with or without a commercially available purification kit or the like.

The present invention can further suitably include: (a) a step of homogenizing the sample; and appropriately freezing or freeze-drying it at 0° C. or lower temperature, or treating it with alcohol or an alcoholic solution, before the step (i). By including this treatment, it becomes possible to collect samples from the same or different examinees at different timings, and thereafter detect an adenoma or cancer in these samples at a same timing.

The alcohol or the alcoholic solution for use in the step (a) can be exemplified by methanol, ethanol, 1-propanol, 2-propanol, and solutions containing at least any one of these alcohols. Preferred examples of the alcohol can include methanol and ethanol. If an alcoholic solution is used, the preferred alcohol concentration is 30% or higher but below 100. More specifically, 70% ethanol can be exemplified. In addition, a water-soluble organic solvent can also be used in the present invention as long as it can provide the same effect as the alcohol or the alcoholic solution does.

In the present invention, it is also possible to detect cDNA of the housekeeping gene instead of the genomic DNA, mRNA, or a protein thereof. In this case, the present invention can further include: (b) a step of producing a cDNA from an RNA which is derived from at least one housekeeping gene among the RNA extracted in the step (i), between the step (i) and the step (ii); and quantifying the cDNA in the step (ii). By so doing, the adenoma or cancer can be detected by means of cDNA detection.

In the step (b), a known method in the art and a commercially available kit (RT-PCR kit or the like) can be employed.

The thus extracted nucleic acid or protein is next subjected to a step for quantifying the sequence constituting itself. If the target of the quantification is a nucleotide sequence, this step can be carried out by, for example: a method in which the concerned nucleotide sequence is hybridized with a sequence complementary thereto, and a label which has been previously attached to the complementary sequence is detected at high sensitivity; a method in which the detection is carried out at high sensitivity with a fluorescent intercalator; or a method in which the concerned nucleotide sequence is amplified by PCR using a primer comprising a sequence complementary to the nucleotide sequence, and its amplification product is appropriately and specifically isolated by gel or capillary electrophoresis or the like, followed by the detection thereof. In addition, there are also enumerated: a one step RT-PCR method in which the RNA-to-cDNA conversion is concurrently performed with PCR; and a NASBA method in which an RNA is amplified directly from an RNA. In these methods, the quantification and the detection become possible by using a fluorescence or enzyme-labeled probe, by using serial dilutions of a restriction enzyme, or by performing electrophoresis. On the other hand, if the target of the quantification is a protein sequence, the concerned step can be carried out by, for example: an enzyme immunoassay, an immunoprecipitation assay, and a sandwich ELISA assay, with use of an antibody that can specifically recognize the protein; a two-dimensional electrophoresis assay; or a western blotting analysis.

In the adenoma or cancer detection method of the present invention, the body fluid sample or the excrement sample has to be appropriately selected according to the type of the adenoma or cancer to be detected. For example: feces can be selected for the detection of colorectal cancer; urine can be selected for the detection of kidney cancer, bladder cancer, or urethra cancer; saliva can be selected for the detection of salivary gland cancer; sputum or pleural effusion can be selected for the detection of throat, trachea, or lung cancer; nasal mucus can be selected for the detection of nasal cancer; lacrimal fluid can be selected for the detection of lacrimal gland cancer; gastric juice can be selected for the detection of gastric cancer; bile can be selected for the detection of liver cancer, gallbladder cancer, or bile duct cancer; pancreatic juice can be selected for the detection of pancreatic cancer; sweat or pus can be selected for the detection of cancer of the skin including sweat glands: cerebrospinal fluid can be selected for the detection of brain tumor; cardiac effusion can be selected for the detection of cardiac tumor; milk can be selected for the detection of breast cancer, vaginal secretion can be selected for the detection of uterine cancer; semen can be selected for the detection of testis cancer; ascites can be selected for the detection of abdominal cancer; amniotic fluid can be selected for the detection of cancer in the placenta or fetus; lymph fluid can be selected for the detection of cancer in the lymph node or lymph fluid; and blood can be selected for the detection of cancer in the blood. Since these body fluid samples or excrement samples are accumulated in the body, it can be considered that normal cells, if existing therein, would die early due to apoptosis which is supposed to occur during the accumulation. On the other hand, cancer cells or genetically abnormal cells within an adenoma accumulated in these body fluid samples or excrement samples are apoptosis-resistant and hardly die unlike the normal cells. For this reason, it is possible to specifically detect adenoma or cancer cells through detection of a housekeeping gene with use of a preserved body fluid sample, excrement sample, or the like which contains body cells. In addition, a housekeeping gene is considered to be three to ten times greater in the abundance and/or the expression level than that of a cancer specific gene (such as a carcinoembryonic antigen gene), by which therefore a highly sensitive detection becomes possible.

In the adenoma or cancer detection method of the present invention, it is preferable to use two or more types of housekeeping genes, or to measure a plurality of quantities of the sequences constituting these housekeeping genes or their expression products at the same time. By having a plurality of housekeeping genes or their expression products as the target of measurement, it becomes possible to detect genes which are not affected by carcinogenesis with higher probability, and thus it becomes possible to more accurately detect the cancer or adenoma through the detection of housekeeping genes derived from cancer cells. An adenoma is often a previous stage of cancer, which is neither invasive nor metastatic. Many adenomas are elevated and thus come off more frequently than normal epithelium mucous membranes. In addition, as described above, adenomas are apoptosis-resistant. Therefore, an adenoma is also detectable through the detection of housekeeping gene(s).

Moreover, the adenoma or cancer detection method of the present invention can further include, after the step (ii):

(iii) a step of measuring the quantity of a sequence constituting at least one tumor gene or an expression product thereof in the extracted nucleic acid or protein, and calculating the concentration of the sequence in the sample; and

(iv) a step of correcting the concentration of the sequence constituting the tumor gene or the expression product thereof that has been calculated in the step (iii), based on the concentration of the sequence constituting the housekeeping gene or the expression product thereof that has been calculated in the step (ii).

Generally, the quantity of the expression product of a housekeeping gene is more abundant than that of a tumor gene per each cell. By utilizing this tendency, the quantity of the expression product of a tumor gene can be detected more quantitatively and more accurately with use of the quantity of the expression product of a housekeeping gene as an internal standard for specimens. Therefore, the accuracy and the sensitivity can be improved in the adenoma or cancer detection which uses the concerned tumor gene. For example, if the expression product of a tumor gene is not detected in a given sample, two scenarios can be considered: a case where the expression product of the tumor gene is truly not contained in the sample; and a case where the expression product of the tumor gene is originally contained therein but nonetheless can not be detected as a result of the occurrence of a problem in the preparation, the preservation, or such a treatment of the sample, the test operation, or the like. The method of the present invention detects not only the expression product of a tumor gene but also the expression product of a housekeeping gene in the same manner. Therefore, for example, if the expression product of the housekeeping gene can not be detected, it can be considered that a problem might have happened in the preparation of a sample or the like and highly possibly yielded false negative results.

When the quantity of the expression product of a housekeeping gene is used as an internal standard, as mentioned above, it is not only possible to merely use the value of the quantity of the expression product of the housekeeping gene alone, but also possible to improve the sensitivity and the accuracy of the detection of an adenoma or cancer, for example, through the correction of the quantity of the expression product of a tumor gene with use of the quantity of the expression product of the housekeeping gene. Specifically, the quantity of the expression product of the tumor gene can be corrected by dividing the quantity of the expression product of the tumor gene by the quantity of the expression product of the housekeeping gene.

In addition, the present invention can provide more reliable test results with particular consideration of the quality, the quantity, or the like of the nucleic acid specimen that has been extracted/purified from a biological sample, in an adenoma or cancer testing method in which feces collected from an examinee is used as a specimen sample and a marker gene for the adenoma or cancer contained in the feces is detected as a target gene (gene serving as a target of detection).

Specifically, the adenoma or cancer testing method of the present invention is a method in which a target gene-derived RNA is detected in RNA extracted/purified from feces that has been collected from an examinee, and the quantity thereof is measured so as to thereby determine whether or not the examinee is affected by the adenoma or cancer, as well as being a method in which the quality or the quantity (concentration) of RNA in use is checked to judge the reliability of the RNA so as to thereby determine that the test result is reliable if the RNA is reliable, or judge that the test result is unreliable if the RNA is unreliable.

In the claims and the specification of this application, the term “RNA is reliable” means that the RNA is adequate enough in the quality or the quantity to provide highly reliable test results. The term “reliable RNA” means, for example, an RNA without a problem in the quality and the quantity of feces which provided the RNA, the method for extracting and purifying the RNA from the feces, the degree of decomposition of the purified RNA, or the degree of purification thereof (proportion of mixed impurities such as salts and proteins). Conversely, the term “RNA is unreliable” means that the RNA is not adequate enough in the quality or the quantity and that the test results given by this RNA are not reliable. The term “unreliable RNA” means, for example, an RNA with a high possibility of a deficiency in the quality and the quantity of feces which provided the RNA, an error operation of the method for extracting and purifying the RNA from the feces, an advanced decomposition of the purified RNA, or severe contamination by impurities such as salts and proteins.

In the present invention, the term “gene-derived RNA” means an RNA transcribed from the full length or a part of the genomic DNA of a gene, and may be an mRNA of the gene, or a part (fragment) of the mRNA.

In addition, in the present invention, the term “target gene” means a marker gene for an adenoma or cancer. Here, the “marker gene for an adenoma or cancer” is not specifically limited as long as a determination on whether or not the examinee is affected by the adenoma or cancer can be made through an analysis of the presence or absence of the expression of the gene in feces or an analysis of the quantity of the expression level thereof. Such a marker gene can be appropriately determined with consideration of the type of the adenoma or cancer, or the like. As for the target gene in the present invention, genes known as adenoma markers or cancer markers can be employed. Such a marker gene can be enumerated by a gene which is specifically expressed in adenoma or cancer cells, a gene in which a mutation such as nucleotide insertion, deletion, substitution, duplication, inversion, or splicing variant (isoform) arises from the development of an adenoma or carcinogenesis of cells, and the like. The target gene in the present invention is preferably a gene selected from the group consisting of cyclooxygenase 2 (COX2), matrix metallopeptidase 7 (MMP7), and SNAIL.

Unlike other biological samples, feces contain an extreme amount of impurities, where nucleic acids can be easily decomposed depending on the preservation condition of the feces before the nucleic acid extraction. Moreover, the quantity of enterobacteria is so large and the quantity of nucleic acids of human-derived cells is so small that the extraction and the purification of nucleic acids are very difficult as compared to other biological samples. In addition, feces are heterogeneous. That is, wide varieties of components are unevenly present, and adenoma or cancer cells are also unevenly present therein. For this reason, even if specimens are collected from the same feces for use in a test, the test result may fluctuate due to the difference in the position where the specimens are collected.

In this manner, if feces is used as a specimen, the quality of the extracted/purified nucleic acid specimen for use in the test will impose a greater influence on the test result than in the case where blood or such other biological sample is used. For this reason, it is necessary for the accurate test to detect or quantify a standard gene upon the provision that the quality and the quantity of the extracted/purified nucleic acid specimen meet the criteria. In the present invention, the quality and the quantity of a nucleic acid specimen that is extracted/purified from feces are measured, and based on these results it is judged whether or not the obtained detection result of the target gene is reliable. Therefore, false negatives and false positives can be remarkably reduced, and more reliable test results can be obtained.

For example, if the specimen is collected from an inappropriate position of the feces, if nucleic acids are decomposed in the feces before the RNA extraction, or if any error occurs during the RNA extraction and purification operations, the total quantity of RNA extracted/purified from the feces will decrease. In addition, if such an RNA is used for the detection and the quantification of the target gene-derived RNA, a reliable result will be hardly given. For example, if such an RNA is used: even if the target gene-derived RNA is detected, it is highly possible that the result is false positive; and if conversely the target gene-derived RNA is not detected, it is possible that the result is false negative.

Therefore, the total quantity of RNA extracted from feces and purified as a solution (hereunder, may be denoted by the “extracted/purified RNA from feces”) can be measured, and if this total quantity is smaller than a previously determined given threshold, the yielded RNA can be judged to be unreliable (not adequate as a sample) and the detection result of the target gene obtained with use of this RNA (that is, the test result) can also be judged to be unreliable. Conversely, if the total quantity of the extracted/purified RNA from feces is equal to or larger than the threshold, the yielded RNA can be judged to be reliable (adequate as a sample) and the detection result of the target gene obtained with use of this RNA can also be judged to be reliable. Since the majority of the extracted/purified RNA from feces is derived from enterobacteria (bacteria), if the total quantity of the extracted/purified RNA which contains bacteria-derived RNA is used as an index, it becomes possible to examine the reliability (sample adequacy) of the yielded RNA more accurately and readily than the case where only the quantity of human adenoma or cancer cell-derived RNA is used as an index.

When the total quantity of the extracted/purified RNA from feces is used as an index of the RNA reliability, the threshold serving as the criterion can be appropriately determined with consideration of the quantity of feces supplied to the RNA extraction and purification, the RNA quantification method, or the like. For example, when RNA is extracted/purified from 0.5 g of feces, the threshold is preferably not less than 5 μg, and more preferably about 100 μg.

Moreover, it is also possible to use the RNA concentration of the RNA solution as an index of the RNA reliability, instead of the total quantity of the extracted/purified RNA from feces. In this case, the threshold serving as the criterion can be appropriately determined with consideration of the quantity of feces supplied to the RNA extraction and purification, the RNA quantification method, or the like. For example, when RNA is extracted/purified from 0.5 g of feces, the threshold is preferably not lower than 10 ng/μL, and more preferably about 100 ng/μL. That is, similarly to the case where the total quantity of RNA is used as an index, if the RNA concentration of the RNA solution extracted from feces is lower than 10 ng/μL, the yielded RNA can be judged to be unreliable and the detection result of the target gene obtained with use of this RNA (that is, the test result) can also be judged to be unreliable. Conversely, if the total quantity of the extracted/purified RNA from feces is equal to or larger than the threshold, the yielded RNA can be judged to be reliable and the detection result of the target gene obtained with use of this RNA can also be judged to be reliable.

As for the quality of the extracted/purified RNA from feces, the index is not specifically limited as long as it is generally employed as an index of the quality of a nucleic acid sample. However, in the present invention, the degree of purification or the degree of decomposition is preferably used as an index. In the present invention, the term “degree of RNA purification” means the proportion of impurities (substances other than RNA) in the extracted/purified RNA. In addition, the term “degree of RNA decomposition” means the proportion of decomposed RNA by nucleases and the like in the extracted/purified RNA. A higher degree of RNA purification and a lower degree of RNA decomposition mean higher RNA quality.

For example, if any error occurs during the RNA extraction and purification operations from feces, a large quantity of impurities from the feces may be left in the extracted and produced RNA. Such impurities often act inhibitingly against the detection or the quantification of the target gene-derived RNA. For this reason, if the target gene-derived RNA is detected and quantified with use of an RNA having a low degree of purification, a reliable result will be hardly given. In fact, it is known that impurities in RNA do inhibit the PCR amplification for the detection of a target gene-derived RNA. Therefore, the degree of purification of the extracted/purified RNA from feces can be measured, and if this degree of purification is out of a previously determined given range, the yielded RNA can be judged to be unreliable and the detection result of the target gene obtained with use of this RNA can also be judged to be unreliable. Conversely, if the degree of purification of the extracted/purified RNA from feces is within the range, the yielded RNA can be judged to be reliable and the detection result of the target gene obtained with use of this RNA can also be judged to be reliable.

The measurement of the degree of RNA purification can be performed by appropriately selecting from known general techniques for use in the measurement of the degree of purification (purity) of a nucleic acid sample. In the present invention, it is preferable to measure the RNA absorbance by using UV, and to use a value resulting from the division of the absorbance at 260 nm by the absorbance at 230 nm (260/230 nm absorbance ratio) or a value resulting from the division of the absorbance at 260 nm by the absorbance at 280 nm (260/280 nm absorbance ratio) as an index of the degree of purification. The concentration ratio of RNA to salts can be understood from the 260/230 nm absorbance ratio. On the other hand, the concentration ratio of RNA to proteins and like substances can be understood from the 260/280 nm absorbance ratio. Therefore, the degree of RNA purification can be understood from these values. Either one or both of these absorbance ratios can be employed.

Specifically, if the 260/230 nm absorbance ratio is smaller than 1.0 or greater than 2.5, because of a high content proportion of salts, the degree of purification can be judged to be insufficient, and the yielded RNA can be judged to be unreliable. Conversely, if the 260/230 nm absorbance ratio is within 1.0 to 2.5, preferably within 1.7 to 2.1, the degree of purification can be judged to be sufficient and the yielded RNA can be judged to be reliable. On the other hand, if the 260/280 nm absorbance ratio is smaller than 1.0 or greater than 2.5, the degree of purification is considered to be insufficient because of contamination by proteins and like substances, and the yielded RNA can be judged to be unreliable. Conversely, if the 260/280 nm absorbance ratio is within 1.0 to 2.5, preferably within 1.7 to 2.1, the degree of purification can be judged to be sufficient and the yielded RNA can be judged to be reliable.

Moreover, if the degree of RNA decomposition is high, that is, if a lot of RNA has been decomposed or fragmented, it is highly possible that the target gene-derived RNA is also decomposed or fragmented. If such an RNA is used for the detection and the quantification of the target gene-derived RNA, a reliable result will be hardly given. Therefore, the degree of decomposition of the extracted/purified RNA from feces can be measured, and if this degree of decomposition is out of a previously determined given range, the yielded RNA can be judged to be unreliable and the detection result of the target gene obtained with use of this RNA can also be judged to be unreliable. Conversely, if the degree of decomposition of the extracted/purified RNA from feces is within the range, the yielded RNA can be judged to be reliable and the detection result of the target gene obtained with use of this RNA can also be judged to be reliable.

The measurement of the degree of RNA decomposition can be performed by appropriately selecting from known general techniques for use in the measurement of the decomposition or fragmentation of a nucleic acid. For example, if a size separation assay is performed through RNA electrophoresis, the quantity per each size of nucleic acid can be understood, and therefore the degree of RNA decomposition can be measured.

In the present invention, it is effective to use bacteria-derived RNA which accounts for a relatively large proportion in the extracted/purified RNA, in particular, to use 23S rRNA and 16S rRNA subunits which are bacterial ribosomal RNAs, as an index to measure the degree of RNA decomposition. For example, when using an undecomposed total RNA, two distinct bands from ribosomal RNAs (bacteria-derived 23S rRNA and 16S rRNA accounting for large proportions in feces) are found in a ratio of about 2:1. In comparison to this, when using a decomposed or fragmented total RNA, bands from the respective ribosomal RNA subunits are dispersed, and these bands are not distinct and are detected in a smeared manner in low molecular size regions. Such a decomposed specimen can not be amplified in a normal manner after performing amplification and detection, and often results in false negatives. For this reason, the specimen for use in the test is desirably less decomposed and less fragmented so that distinct bands can be yielded.

Specifically, if a value resulting from the division of the quantity of a fragment of 23S ribosomal RNA by the quantity of a fragment of 16S ribosomal RNA (23S rRNA/16S rRNA ratio) is within 1.6 to 2.5, preferably within 1.8 to 2.0, the degree of decomposition can be judged to be sufficiently low and the yielded RNA can be judged to be reliable. Conversely, if the 23S rRNA/16S rRNA ratio is smaller than 1.6 or greater than 2.5, the degree of decomposition can be judged to be high and the yielded RNA can be judged to be unreliable.

The “Bioanalyzer” electrophoresis system of Agilent Technologies, which is applicable to RNA electrophoresis, is one of the widely used automatic capillary gel electrophoresis systems in the field of molecular biology (for example, refer to “A microfluidic system for high speed reproducible DNA sizing and quantitation”, Electrophoresis, 200, Vol. 21, No. 1, pp. 128 to 134). Because this system automatically displays the quantification result per each size of nucleic acid on the completion of measurement, the 28S rRNA/18S rRNA ratio (value resulting from the division of the quantity of a fragment of 28S ribosomal RNA by the quantity of a fragment of 18S ribosomal RNA) which is a ribosomal RNA ratio, the 23S rRNA/16S rRNA ratio, and other values of bands, can be understood. Therefore, the degree of decomposition and the degree of purification of RNA of interest can be estimated from the proportion of decomposition or fragmentation of these ribosomal RNAs. The RIN (RNA Integrity Number) value, which is one of the algorithms of this system, is generally used as one of the indexes of the degree of nucleic acid decomposition. With use of this RIN value (range of 1 to 10), it can be said that a higher RIN value (=10) means a lower degree of decomposition and a lower RIN value (=1) means a higher degree of decomposition. RNA derived from a fecal specimen contains lots of impurities and is easily decomposed, which may impose a great influence on the following nucleic acid amplification reaction depending on the proportion of impurities and the degree of decomposition. For this reason, it is one of the effective means to check the RNA quality with use of this RIN value as an index.

RIN values which can indicate a good quality of a fecal specimen-derived RNA were obtained, by which the range of such RIN values was found to be 10 to 4. With an RNA having a RIN value of 1 to 2, the following nucleic acid detection reactions such as a nucleic acid amplification reaction were unsuccessful. Therefore, the yielded RNA was found to be in a bad quality and unreliable. For this reason, regarding the checking of the quality of a fecal specimen-derived RNA, the threshold of the RIN value is preferably set at 3.

Besides, it is also possible to use the content quantity of the standard gene-derived RNA as an index to check the quality of the extracted/purified RNA from feces. If the quantity of the standard gene-derived RNA in the RNA is equal to or above a previously determined given threshold, it can be judged that the collection/preservation of feces and the RNA extraction/purification operations from feces have been done appropriately, the yielded RNA can be judged to be reliable, and the detection result of the target gene obtained with use of this RNA can also be judged to be reliable. Conversely, if the quantity of the standard gene-derived RNA in the RNA is below a previously determined given threshold, the yielded RNA can be judged to be unreliable and the detection result of the target gene obtained with use of this RNA can also be judged to be unreliable.

The standard gene is not specifically limited as long as an RNA derived from this gene can be expected to exist in the RNA from feces, although a human gene is preferable. With use of a human gene as a standard gene, it is possible to check the presence of human-derived cells in feces that has been used for the test. In addition, the detectability of the human gene-derived RNA which accounts for a very small proportion in the RNA indicates that the RNA is in a very good quality. Therefore, the reliability of the detection result of the target gene can be more improved.

In the present invention, the standard gene is preferably a housekeeping gene or an epithelial cell-specific gene. As for the housekeeping gene, genes mentioned above can be employed. On the other hand, in the present invention, the term “epithelial cell-specific gene” means a gene that is specifically expressed in epithelial cells. The term “specifically expressed in epithelial cells” does not require completely no expression in non-epithelial cells, but may refer to remarkably higher expression levels in epithelial cells than in other cells. Such an epithelial cell-specific gene can be exemplified by carcinoembryonic antigen genes, cell adhesion factor genes, mucin genes, and cytokeratin genes. The carcinoembryonic antigen genes can be exemplified by a carcinoembryonic antigen (CEA) gene, the cell adhesion factor genes can be exemplified by an epithelial cell adhesion molecule (EpCAM) gene, the mucin genes can be exemplified by a mutin 2 (MUC2) gene, a mutin 3 (MUC3) gene, and a mutin 4 (MUC4) gene, and the cytokeratin genes can be exemplified by a keratin 7 (CK7) gene, a keratin 19 (CK19) gene, and a keratin 20 (CK20) gene. In the present invention, as for the standard gene, it is preferable to use a gene selected from the group consisting of glyceraldehyde-3-phosphate dehydrogenase (GAPDH), 18S ribosomal RNA, 28S ribosomal RNA, β actin, β2 microglobulin, hypoxanthine phosphoribosyl transferase 1, ribosomal protein large P0, peptidylpropyl isomerase A (cyclosporin A), cytochrome C, phosphoglycerate kinase 1, (β-glucuronidase, TATA box binding factor, transferrin receptor, HLA-A0201 heavy chain, ribosomal protein L19, α tubulin, β tubulin, γ tubulin, ATP synthetase, eukaryotic translation elongation factor 1 gamma (EEF1G), succinate dehydrogenase complex (SDHA), aminolevulinic acid synthase 1 (ALAS1), ADP-ribosylation factor 6, endonuclease G (ENDOG), peroxisomal biogenesis factor (PEX), carcinoembryonic antigen (CEA), epithelial cell adhesion molecule (EpCAM), mutin 2 (MUC2), mutin 3 (MUC3), mutin 4 (MUC4), keratin 7 (CK7), keratin 19 (CK19), and keratin 20 (CK20). It is more preferable to use (β2 microglobulin, glyceraldehyde-3-phosphate dehydrogenase (GAPDH), or CEA, as these genes can be satisfactorily and stably detected from feces.

For example, a standard gene-derived RNA originated from human can be highly sensitively detected through amplification together with a target gene-derived RNA. In addition, since in general housekeeping genes and epithelial cell-specific genes are kinds of genes which are expressed at constant levels at all times, if such a gene is employed as a standard gene, the detectability of the standard gene-derived RNA can be used as an index of the testing process. In particular, when both the target gene-derived RNA and the standard gene-derived RNA are to be detected by PCR, it is also preferable to perform multiplex PCR.

Specifically, the adenoma or cancer testing method of the present invention is a method for testing an adenoma or cancer with use of a marker gene for the adenoma or cancer (target gene), which comprises the following steps, provided that the steps (B), (C), (D), and (E) may be carried out in the order of the steps (D), (E), (B), and (C), the steps (B), (D), (C), and (E), the steps (B), (D), (E), and (C), the steps (D), (B), (E), and (C), or the steps (D), (B), (C), and (E):

(A) a step of extracting an RNA contained in feces that has been collected from an examinee, and purifying it as an RNA solution;

(B) a step of measuring the quantity of a target gene-derived RNA in the RNA solution obtained in the step (A);

(C) a step of comparing the quantity of the target gene-derived RNA obtained in the step (B) with a preset threshold, to determine whether or not the examinee is affected by the adenoma or cancer;

(D) a step of measuring one or more items selected from the group consisting of the degree of RNA purification, the degree of RNA decomposition, the RNA concentration, and the quantity of a standard gene-derived RNA, in the RNA solution obtained in the step (A);

(E) a step of judging the reliability of the RNA in the RNA solution obtained in the step (A), based on the value obtained in the step (D); and

(F) a step of judging that the determination of the step (C) is reliable if the RNA is judged to be reliable in the step (E), and judging that the determination of the step (C) is unreliable if the RNA is judged to be unreliable in the step (E).

Hereunder is a description of the respective steps.

First, as for the step (A), the RNA contained in feces that has been collected from an examinee is extracted, and purified as an RNA solution. The method for extracting and purifying RNA from feces is not specifically limited, and any known method in the art can be employed with or without a commercially available purification kit or the like. Before proceeding to the next step, the concentration of the RNA in the RNA solution obtained in the step (A) (hereunder, may be simply referred to as the “RNA of the step (A)”) may be measured. The method for measuring the RNA concentration is not specifically limited and any known method in the art such as absorption spectroscopy can be employed.

As for the step (B), the quantity of a target gene-derived RNA in the RNA solution obtained in the step (A) is measured. In the step (B), the method for measuring the quantity of the target gene-derived RNA is not specifically limited, and can be performed by appropriately selecting from known general techniques for use in the measurement of the quantity of a specific nucleic acid. In the present invention, the term “measurement of the quantity of an RNA” does not mean a strict quantification, and may include a semiquantitative measurement, and a kind of measurement which can allow a quantitative comparison with a given threshold or the like. For example, with known techniques in the art, it is possible to detect the target gene-derived RNA, and to calculate the quantity thereof from the thus obtained detection result with reference to a correction curve prepared by detection results of a control sample at known concentrations. The method for detecting the target gene-derived RNA is not specifically limited, and any known method in the art can be employed. For example, the detection can be done by a hybridization method with use of a probe which can hybridize with the target gene-derived RNA, or a method using a nucleic acid amplification reaction with use of a primer which can hybridize with the target gene-derived RNA and a polymerase. In addition, a commercially available purification kit or the like can also be employed.

Since the quantity of the target gene-derived RNA is very small, it is preferable to measure it by a method which employs a nucleic acid amplification reaction. For example, the target gene-derived RNA can be detected and its quantity can be measured by synthesizing a cDNA through a reverse transcription reaction (RT-PCR: reverse transcriptase-polymerase chain reaction) of the total amount of a part of the RNA in the RNA solution obtained in the step (A), and nucleic acid amplification with use of the obtained cDNA as a template. In addition, the detection and the quantification of the target gene-derived RNA can be concurrently carried out with ease by performing semiquantitative PCR such as real-time PCR as the nucleic acid amplification.

Moreover, the measurement of the quantity of target gene-derived RNA can also be carried out after normalization (adjustment to a previously determined concentration) of the RNA solution obtained in the step (A). For example, it is possible to perform, after normalization of the RNA solution obtained in the step (A), the reverse transcription reaction, and nucleic acid amplification such as PCR or real-time PCR with use of the obtained cDNA as a template. The concentration to be normalized can be appropriately determined with consideration of the operation to detect the target gene-derived RNA, and the like.

After the step (B), the step (C) is carried out by comparing the quantity of the target gene-derived RNA obtained in the step (B) with a preset threshold, to determine whether or not the examinee is affected by the adenoma or cancer. For example, when the target gene is a kind of gene whose expression level increases as the development of an adenoma or carcinogenesis proceeds, it is possible to determine that the examinee is affected by the adenoma or cancer if the quantity of the target gene-derived RNA obtained in the step (B) is larger than a preset threshold, or to determine that the examinee is unaffected by the adenoma or cancer if the quantity is smaller than the threshold. Conversely, when the target gene is a kind of gene whose expression level decreases as the development of an adenoma or carcinogenesis proceeds, it is possible to determine that the examinee is affected by the adenoma or cancer if the quantity of the target gene-derived RNA obtained in the step (B) is smaller than a preset threshold, or to determine that the examinee is unaffected by the adenoma or cancer if the quantity is larger than the threshold. The threshold can be appropriately set by considering the kind of the target gene, the kind of the detection method, and the like, or by performing a necessary preliminary test, and the like, by those skilled in the art. For example, an appropriate threshold can be set in advance by comparing the quantity of the target gene in feces collected from a healthy subject who is not affected by the adenoma or cancer with the quantity of the target gene in feces collected from an examinee who has been found to be affected by the adenoma or cancer.

Moreover, the index value(s) for determining the reliability of the RNA of the step (A) is(are) measured in the step (D), and the sample adequacy of the RNA is judged in the step (E). Specifically, one or more items selected from the group consisting of the degree of RNA purification, the degree of RNA decomposition, the RNA concentration, and the quantity of the standard gene-derived RNA, in the RNA solution obtained in the step (A) is(are) measured, and based on the(se) value(s) a judgement is made on whether or not the RNA is reliable. The judgement on the reliability of the RNA of the step (A) can be made more strictly by measuring a plurality of these values and employing a plurality of indexes. The measurements of the degree of RNA purification, the degree of RNA decomposition, the RNA concentration, and the quantity of the standard gene-derived RNA, and the judgement on the reliability can be carried out in the same manner as mentioned above.

Finally, the step (F) is carried out by judging that the determination of the step (C) is reliable if the RNA used in the measurement of the quantity of the target gene-derived RNA is judged to be reliable in the step (E), or conversely judging that the determination of the step (C) is unreliable if the RNA is judged to be unreliable in the step (E). By so doing, more reliable test results can be obtained.

The detection of the target gene-derived RNA in the steps (B) and (C) and the measurement of the index value(s) for determining the reliability of the RNA in the steps (D) and (E) can be performed either in this order or vice versa. That is, the steps (B), (C), (D), and (E) can be carried out in any order as long as the step (B) comes before the step (C) and the step (D) comes before the step (E). Specifically, the steps (B), (C), (D), and (E) may also be in the order of the steps (D), (E), (B), and (C), the steps (B), (D), (C), and (E), the steps (B), (D), (E), and (C), the steps (D), (B), (E), and (C), or the steps (D), (B), (C), and (E).

Moreover, if the step (E) is carried out before the step (B) or (C), and if the RNA of the step (A) is judged to be unreliable, the test can be terminated without performing the step (B) or (C) thereafter. In addition, if the RNA is judged to be unreliable in this way, a retest or the like can be carried out if necessary.

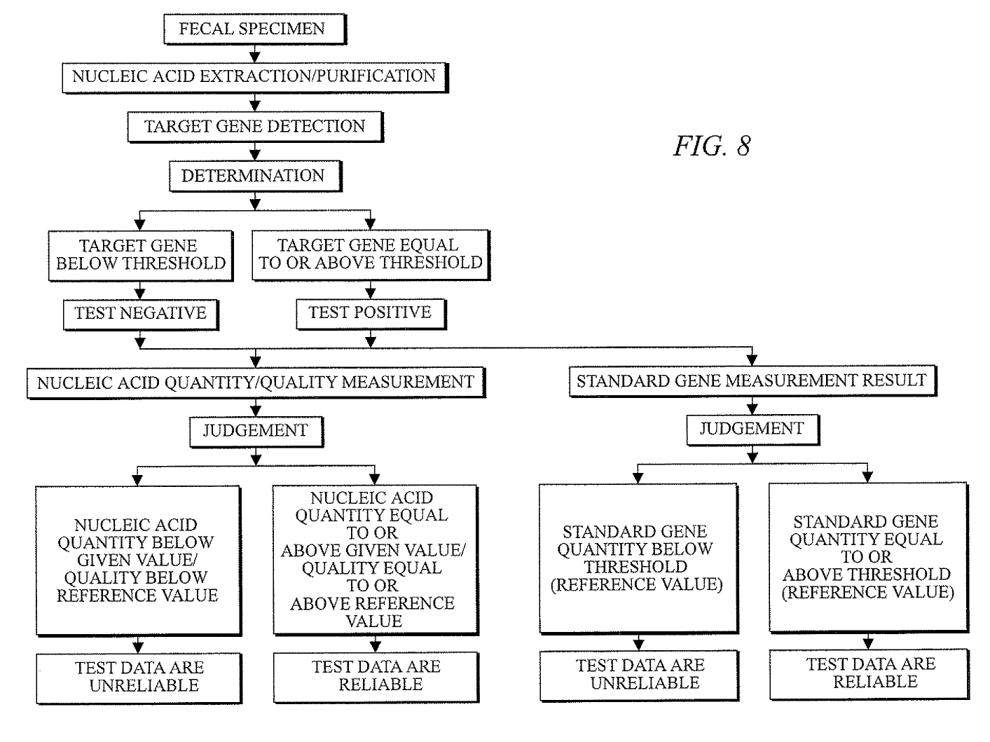

FIG. 8 to FIG. 10 respectively show flowcharts of embodiments of the adenoma or cancer testing method of the present invention, in the case where the target gene is a kind of gene whose expression level increases as the development of an adenoma or carcinogenesis proceeds. It is needless to say that the present invention is not to be limited to these embodiments.

FIG. 8 shows a method in which the RNA reliability is judged after the measurement of the quantity of the target gene-derived RNA and the determination on whether or not the examinee is affected by an adenoma or cancer. First, RNA is extracted from feces (fecal specimen) that has been collected from an examinee, and purified as a solution (step (A)). The quantity of the target gene-derived RNA in the obtained RNA is measured (step (B)). The obtained quantity of the target gene-derived RNA is compared with a preset threshold, and if the quantity of the target gene-derived RNA is larger than the preset threshold (in the flowchart “TARGET GENE EQUAL TO OR ABOVE THRESHOLD”), the examinee is determined to be affected by the adenoma or cancer (in the flowchart, “TEST POSITIVE”), while if the quantity is smaller than the threshold (in the flowchart, “TARGET GENE BELOW THRESHOLD”), the examinee is determined to be unaffected by the adenoma or cancer (in the flowchart, “TEST NEGATIVE”) (step (C)). Then, the concentration (the quantity), the quality (the degree of purification or the degree of decomposition), and the like of the RNA are measured (step (D), in the flowchart, “NUCLEIC ACID QUANTITY/QUALITY MEASUREMENT”), by which the reliability of the RNA obtained in the step (A) is judged (step (E)). If the measured RNA concentration is lower than a previously determined threshold (in the flowchart, “NUCLEIC ACID QUANTITY BELOW GIVEN VALUE”), or if the measured degree of RNA purification or the measured degree of RNA decomposition is out of a previously determined range (in the flowchart, “NUCLEIC ACID QUALITY BELOW REFERENCE VALUE”), the RNA is judged to be unreliable, and the result on the test positive or negative is judged to be unreliable (step (F)). On the other hand, if the measured RNA concentration is equal to or higher than the previously determined threshold (in the flowchart, “NUCLEIC ACID QUANTITY EQUAL TO OR ABOVE GIVEN VALUE), or if the measured degree of RNA purification or the measured degree of RNA decomposition is within the previously determined range (in the flowchart, “NUCLEIC ACID QUALITY EQUAL TO OR ABOVE REFERENCE VALUE”), the RNA is judged to be reliable, and the result on the test positive or negative is judged to be reliable (step (F)). It is also possible such that in the RNA quality check, the quantity of the standard gene in the RNA is measured (step (D), in the flowchart, “STANDARD GENE MEASUREMENT RESULT”) and compared with a preset threshold, and if the quantity of the standard gene-derived RNA is larger than the threshold, (in the flowchart, “STANDARD GENE QUANTITY EQUAL TO OR ABOVE THRESHOLD (REFERENCE VALUE)”), the result on the test positive or negative is judged to be reliable, while if the quantity is smaller than the threshold (in the flowchart, “STANDARD GENE QUANTITY BELOW THRESHOLD (REFERENCE VALUE)”), the result on the test positive or negative is judged to be unreliable (step (F)).

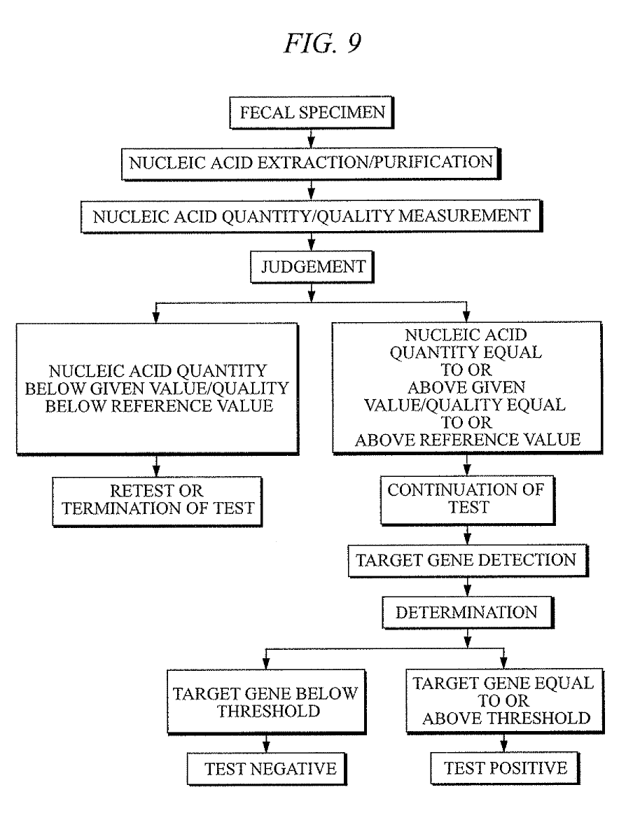

FIG. 9 shows a method in which the RNA reliability is judged before the measurement of the quantity of the target gene-derived RNA. The captions in this flowchart are the same as those of FIG. 8. First, RNA is extracted from feces (fecal specimen) that has been collected from an examinee, and purified as a solution (step (A)). Then, the concentration (the quantity), the quality (the degree of purification or the degree of decomposition), and the like of the RNA are measured (step (D)), by which the reliability of the RNA obtained in the step (A) is judged (step (E)). That is, if the measured RNA concentration is lower than a previously determined threshold, or if the measured degree of RNA purification or the measured degree of RNA decomposition is out of a previously determined range, the RNA is judged to be unreliable, and either a retest is carried out or the test is terminated without performing the following steps. On the other hand, if the measured RNA concentration is equal to or higher than the previously determined threshold, or if the measured degree of RNA purification or the measured degree of RNA decomposition is within the previously determined range, the RNA is judged to be reliable, and the test is carried on. Then, the quantity of the target gene-derived RNA in the RNA is measured (step (B)). The obtained quantity of the target gene-derived RNA is compared with a preset threshold, and if the quantity of the target gene-derived RNA is larger than a preset threshold, the examinee is determined to be affected by the adenoma or cancer, while if the quantity is smaller than the threshold, the examinee is determined to be unaffected by the adenoma or cancer (step (C)), and these determination results are judged to be highly reliable (step (F)).

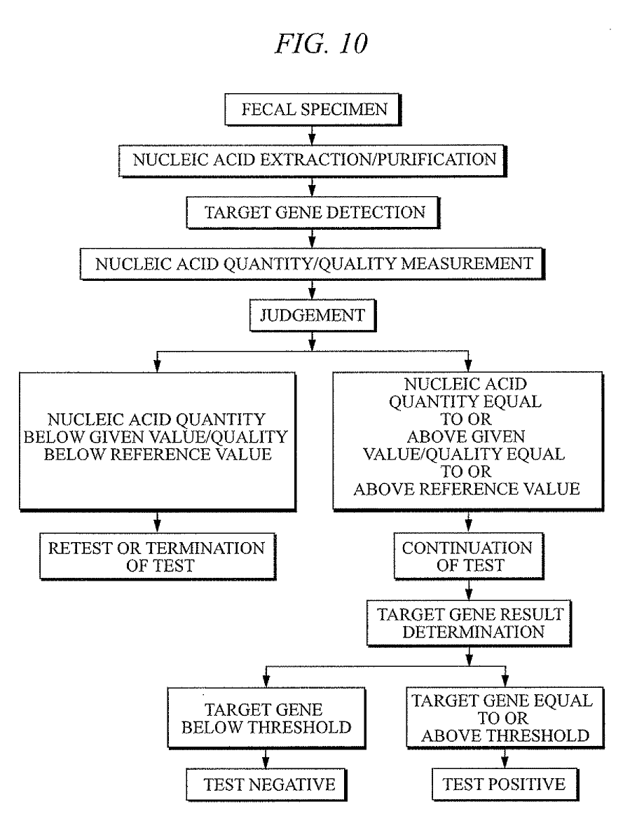

FIG. 10 shows a method in which the judgement is made after the measurement of the quantity of the target gene-derived RNA and the measurement of the indexes of the RNA quantity and quality. The captions in this flowchart are the same as those of FIG. 8. First, RNA is extracted from feces (fecal specimen) that has been collected from an examinee, and purified as a solution (step (A)). The quantity of the target gene-derived RNA in the obtained RNA is measured (step (B)), and then subsequently the concentration (the quantity), the quality (the degree of purification or the degree of decomposition), and the like of this RNA are measured (step (D)), by which the reliability of the RNA is judged (step (E)). If the measured RNA concentration is lower than a previously determined threshold, or if the measured degree of RNA purification or the measured degree of RNA decomposition is out of a previously determined range, the RNA is judged to be unreliable, and either a retest is carried out or the test is terminated without performing the following steps including the judgement on the test results of the target gene-derived RNA. On the other hand, if the measured RNA concentration is equal to or higher than the previously determined threshold, or if the measured degree of RNA purification or the measured degree of RNA decomposition is within the previously determined range, the RNA is judged to be reliable, and the test is carried on. Then, the measured quantity of the target gene-derived RNA is compared with a preset threshold, and if the quantity of the target gene-derived RNA is larger than the threshold, the examinee is determined to be test positive, while if the quantity is smaller than the threshold, the examinee is determined to be test negative (step (C)), and these determination results are judged to be highly reliable (step (F)).

In the present invention, the determination on whether or not the examinee is affected by the adenoma or cancer (the determination on whether the examinee is test positive or test negative) can also be made by using the ratio of the quantity of the target gene-derived RNA to the quantity of the standard gene-derived RNA as a reference. That is, a value resulting from the division of the quantity of the target gene-derived RNA in the extracted/purified RNA in the step (A) by the quantity of the standard gene-derived RNA in the RNA is used as a reference value, and if this reference value is greater than a preset threshold, the examinee is determined to be affected by the adenoma or cancer, while if the value is smaller than the preset threshold, the examinee is determined to be unaffected by the adenoma or cancer. In this way, by using the ratio of the quantity of the target gene-derived RNA to the quantity of the standard gene-derived RNA as a reference, the adenoma or cancer can be more accurately detected than in the case where only the quantity of the target gene-derived RNA is used as a reference.

In this way, since the adenoma or cancer testing method of the present invention combines indexes of the testing process, in particular, indexes related to the reliability of RNA collected from feces, the adenoma or cancer can be tested with high accuracy. In addition, heretofore, it has not been easy to understand the inappropriate point in the testing process if a false positive or a false negative occurs due to a deterioration in the quality or the quantity of the nucleic acid sample, and in the case of retesting, it has been often necessary to carry out the whole process again from the beginning, which takes time, labor, and cost, and which involves complicated procedures. In contrast, in the adenoma or cancer testing method of the present invention, the adequacy of the testing process can be checked at stages during the test without fail, and the determination on the necessity for the retest can be readily made at low cost.

Next is a more detailed description of the present invention with reference to examples. However, the present invention is not to be limited to the following examples.

### Example 1

### (Methods)

Feces was collected from a healthy subject, 6 g of which was placed in a 15 ml tube (manufactured by FALCON) and evenly mixed. Then, the mixture was divided into six samples of 1 g each. Of these, one sample was frozen at −20° C. for the analysis to come and preserved at −20° C. as it was. The remaining five samples were used right after the sampling, one of which was used as it was, and the remaining four were each added with 1 ml of the colon cancer cell line CCK-81. These samples were then respectively added with 5 ml of PBS, and mixed with a homogenizer to yield homogenized products. The homogenized samples were centrifuged at 4000×g for 10 minutes. Their supernatants were taken and RNA was extracted therefrom with the QIAGEN's RNeasy kit. A portion of each extracted RNA, 21.5 μl of RNase-free water, 25 μl of 2× TaqMan Universal PCR Master Mix, and 2.5 μl of a primer/probe set respectively for CEA, GAPDH, and 18S rRNA (manufactured by Applied Biosystems) were put in a 0.2 ml PCR tube and mixed therein. The probe used here was a reporter probe having a fluorophore at one end and a quencher at the other end. These mixtures were subjected to nucleic acid amplification with a real-time fluorescence assay under a temperature condition consisting of: one cycle of 95° C. for 20 seconds; and following 40 cycles of 95° C. for 3 seconds and 60° C. for 30 seconds, by using the 7500 Fast system (manufactured by Applied Biosystems). Plasmids containing cDNA of GAPDH, CEA, or 18 S rRNA were used as standard substances for the copy number calculation, and were amplified at the same time.

### (Results)

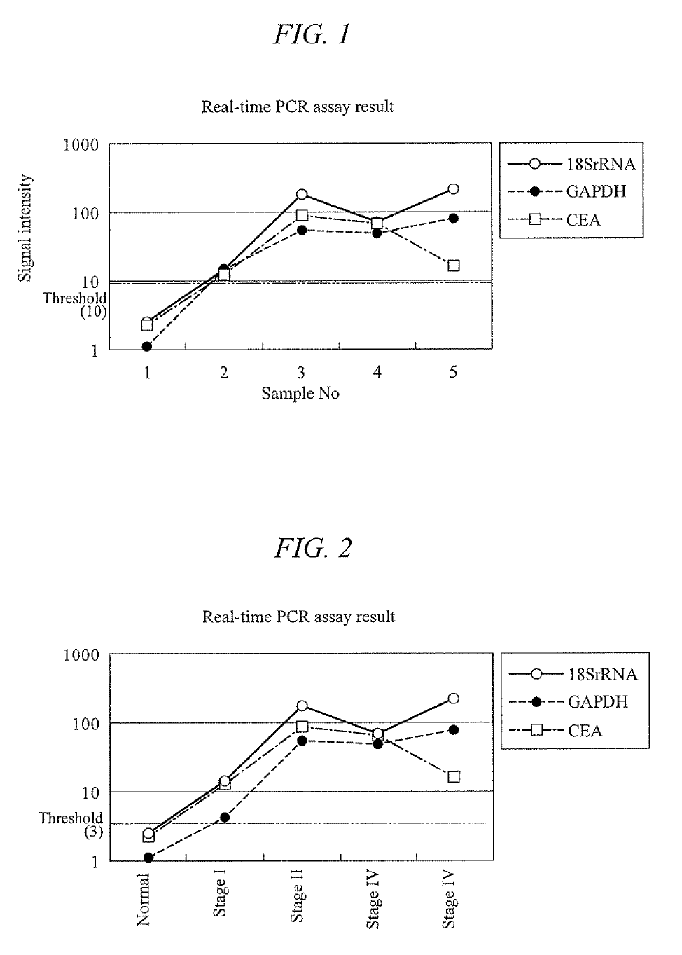

Signals from the nucleic acid amplification were obtained. FIG. 1 shows the summarized results of the fluorescence intensities from the patient samples (samples with the cancer cell line) and the healthy subject sample (sample without the cancer cell line). In this graph, the sample No. 1 represents feces of the healthy subject and the sample Nos. 2 to 5 represent feces of the healthy subject including the colon cancer cell line CCK-81. The data of 18S rRNA are indicated by open circles, the data of GAPDH are indicated by solid circles, and the data of CEA are indicated by open squares.

In the case of 1 g of the feces of the healthy subject alone, all detection values of the housekeeping genes of 18S rRNA, GAPDH, and CEA were small at 5 or under in terms of the relative value. On the other hand, in the samples with the cancer cell line, the detection values were over 10. Therefore, the threshold was able to be set at around 10 in the relative value. The 18S rRNA and GAPDH were detected better than CEA (for example, the sample No. 5 showed higher detection values of 18S rRNA and GAPDH than that of CEA). These results showed that, with use of these housekeeping genes, it was possible to determine that values equal to or above the threshold of 10 pg/μl per total RNA were positive and values below this threshold were negative. By so doing, the presence of small quantities of normal cells which generated these housekeeping genes was found in the healthy subject, while the presence of large quantities of cancer cells which generated these housekeeping genes was proven in the cancer cell line.

As a result of the determination, the sample No. 1 had the 18S rRNA, GAPDH, and CEA values below the threshold of 10 and thus was determined to be normal, while the sample Nos. 2 to 5 had at least one of these values above the threshold of 10, which were twice or more than that of the normal sample No. 1, and thus were determined to be highly possibly cancerous.

### Example 2

Feces were collected from one healthy subject and four colon cancer patients (stages I to IV), and 1 g of each sample was respectively placed in a 15 ml tube (manufactured by FALCON). Right after the sampling, the specimen samples were frozen at −80° C., and then added with an acid phenol-guanidine-chloroform solution. These samples were homogenized with a homogenizer. The homogenized samples were centrifuged at 4000×g for 10 minutes. Their supernatants were taken and RNA was extracted therefrom with the QIAGEN's RNeasy kit. A portion of each extracted RNA, 21.5 μl of RNase-free water, 25 μl of 2× TaqMan Universal PCR Master Mix, and 2.5 μl of a primer/probe set respectively for CEA, GAPDH, and 18S rRNA (manufactured by Applied Biosystems) were put in a 0.2 ml PCR tube and mixed therein. The probe used here was a reporter probe having a fluorophore at one end and a quencher at the other end. These mixtures were subjected to nucleic acid amplification with a real-time fluorescence assay under a temperature condition consisting of: one cycle of 95° C. for 20 seconds; and following 40 cycles of 95° C. for 3 seconds and 60° C. for 30 seconds, by using the 7500 Fast system (manufactured by Applied Biosystems). Plasmids containing cDNA of GAPDH, CEA, or 18S rRNA were used as standard substances for the copy number calculation, and were amplified at the same time.

### (Results)

Signals from the nucleic acid amplification were obtained. FIG. 2 shows the summarized results of the relative values of the fluorescence intensities from the healthy subject sample and the colon cancer patient samples. In this graph, the sample on the most left represents the healthy subject sample (denoted by “Normal” in the graph), and the remaining samples represent the colon cancer patient samples at respective stages. The data of 18S rRNA are indicated by open circles, the data of GAPDH are indicated by solid circles, and the data of CEA are indicated by open squares. CEA is usually used as a marker for colon cancer, and 18S rRNA and GAPDH are usually used as housekeeping genes.

In the case of the healthy subject sample, all detection values of the housekeeping genes of 18S rRNA and GAPDH were small at under 3 in terms of the relative value. On the other hand, in the colon cancer patient samples at all stages, the detection values were over 3. Therefore, the threshold was able to be set at around 3 in the relative value. These results suggested that, in the test of this time, if these housekeeping genes were used, it was possible to determine that those showing equal or greater values than the threshold of 3 in terms of the relative value of the fluorescence intensity were positive and those showing smaller values than this threshold were negative. Since this threshold varies depending on the concentration of the control plasmid to be used, the threshold can be set in advance by a preliminary experiment. In addition, 18S rRNA and GAPDH were detected better than CEA (for example, the stage IV sample on the most right showed a lower CEA detection value than 18S rRNA and GAPDH detection values). From these results, the presence of small quantities of normal cells which generated these housekeeping genes was found in the healthy subject, while the presence of large quantities of cancer cells which generated these housekeeping genes was found in the cancer patients, regarding feces excreted in a normal manner.

As a result of the determination, the healthy subject sample had the 18S rRNA, GAPDH, and CEA values below the threshold of 3 and thus determined to be test negative, while the four colon cancer samples (stages I to IV) had at least one of the 18S rRNA, GAPDH, and CEA values above the threshold of 3 and thus determined to be test positive.

### Example 3

Feces were collected from four healthy subjects, one adenoma carrier, and seven colon cancer patients (stages I to IV), and 1 g of each sample was respectively placed in a 15 ml tube (manufactured by FALCON). Right after the sampling, the specimen samples were frozen at −80° C., and then added with an acid phenol-guanidine-chloroform solution. These samples were homogenized with a homogenizer. In the same manner as that of Example 2, RNA was extracted from the homogenized samples. A portion of each extracted RNA, 21.5 μl of RNase-free water, 25 μl of 2× TaqMan Universal PCR Master Mix, and 2.5 μl of a primer/probe set respectively for CEA and GAPDH (manufactured by Applied Biosystems) were put in a 0.2 ml PCR tube and mixed therein. The probe used here was a reporter probe having a fluorophore at one end and a quencher at the other end. These mixtures were subjected to nucleic acid amplification with a real-time fluorescence assay under a temperature condition consisting of one cycle of 95° C. for 20 seconds; and following 40 cycles of 95° C. for 3 seconds and 60° C. for 30 seconds, by using the 7500 Fast system (manufactured by Applied Biosystems). Plasmids containing cDNA of GAPDH, CEA, or 18S rRNA were used as standard substances for the copy number calculation, and were amplified at the same time.

### (Results)

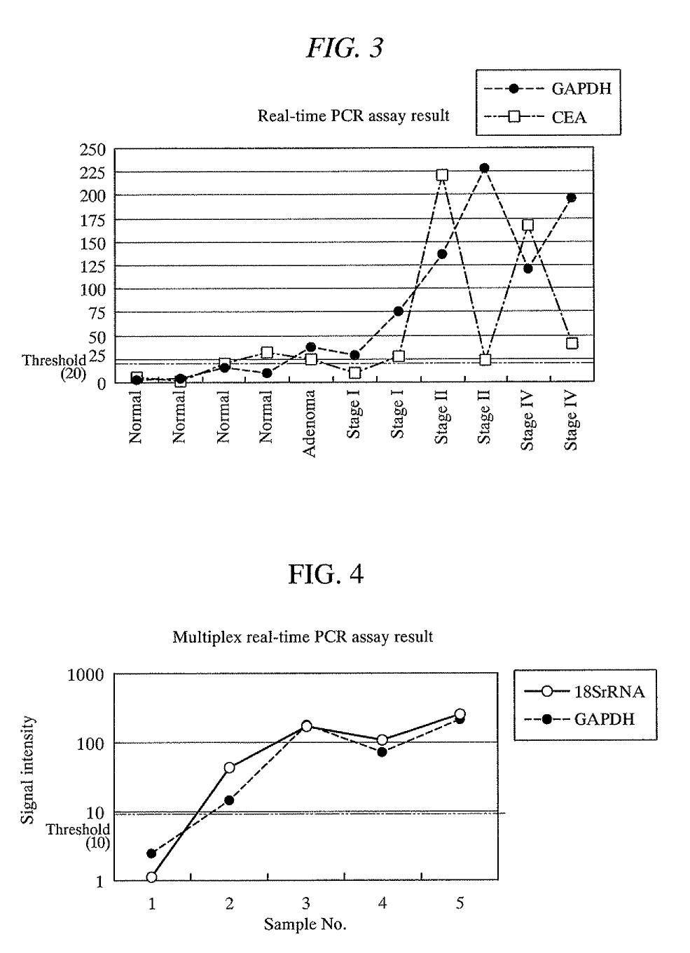

Signals from the nucleic acid amplification were obtained. FIG. 3 shows the summarized results of the relative values of the fluorescence intensities from the healthy subject samples, the adenoma patient sample, and the colon cancer patient samples. In this graph, four samples from the left represent the healthy subject samples (denoted by “Normal” in the graph), and the remaining seven samples represent the adenoma or colon cancer patient samples. The data of GAPDH are indicated by solid circles and the data of CEA are indicated by open squares.

In the experiment at this time, all detection values of the housekeeping gene of GAPDH were small at under 20 in the relative value in the cases of the healthy subject samples, while the detection values thereof were over 20 in the relative value in the fecal samples of the adenoma or colon cancer patients. Therefore, the threshold was able to be set at around 20 in the relative value. Since the threshold varies depending on the concentration of the plasmid control, the threshold can be set in advance by a preliminary experiment having a constant concentration of the plasmid control. GAPDH was detected better than CEA (for example, the adenoma specimen at the fifth from the left, the stage I specimens at the sixth and the seventh from the left, the stage II specimen at the ninth from the left, and the stage IV specimen on the most right clearly showed lower CEA detection values than GAPDH detection values). These results showed that, if the housekeeping gene was used, it was possible to discriminate between normal cases and adenoma/colon cancer cases, more accurately than using CEA. Moreover, it was suggested to be possible to determine that those showing equal or greater values than the threshold of 20 were positive and those showing smaller values than this threshold were negative.

As a result of the determination, the four normal specimens (healthy subjects) had the GAPDH value below the threshold of 20 and thus determined to be test negative, while the adenoma sample and the colon cancer samples (stages I to IV) had the GAPDH value above the threshold of 20 and thus determined to be test positive.

In addition, from the test results of Example 3, the relative values of the fluorescence intensities were higher in the stage II colon cancer cases than in the adenoma or stage I cases, while the stage II and stage IV cases were distributed in an approximately same range. This implies that the value is changed between two steps of: a step including adenoma and stage I case; and a step including stage II and more advanced stages, that is, the relative value of the fluorescence intensity increases in accordance with the degree of progression of colon cancer.

### Example 4

Feces was collected from a healthy subject, 6 g of which was placed in a 15 ml tube (manufactured by FALCON) and evenly mixed. Then, the mixture was divided into six samples of 1 g each. These samples were treated by immersion in 2 ml of 70% alcohol, and preserved at normal temperature. Of these, five samples were prepared by discarding the alcohol, one of which was used as it was, and the remaining four were each added with 1 ml of the colon cancer cell line CCK-81. These samples were then respectively added with 5 ml of PBS, and mixed with a homogenizer to yield homogenized products. The homogenized samples were centrifuged at 4000×g for 10 minutes. Their supernatants were taken and RNA was extracted therefrom with the QIAGEN's RNeasy kit. Using the same samples as those of Example 1, 18S rRNA and GAPDH were amplified at the same time by the multiplex PCR. The probe for 18S rRNA was labeled with FAM and the probe for GAPDH was labeled with VIC. As a result, the respective samples gave similar results as those of FIG. 1, showing that the same results can be obtained by the multiplex assay (not shown in a graph). Accordingly, this method is useful because the cancer detection accuracy is improved by detecting a plurality of markers at the same time, and thereby the cost and the sample quantity can be saved.

### Example 5

Feces was collected from a healthy subject, 5 g of which was placed in a 15 ml tube (manufactured by FALCON) and evenly mixed. Then, the mixture was divided into five samples of 1 g each. These samples were treated by immersion in 2 ml of 70% alcohol, and preserved at normal temperature. Of these, one sample (No. 1) was prepared by discarding the alcohol, and the remaining four samples (Nos. 2 to 5) were prepared by discarding the alcohol and then adding with 1 ml of the colon cancer cell line CCK-81. These samples were then respectively added with 5 ml of PBS, and mixed with a homogenizer to yield homogenized products. The homogenized samples were centrifuged, and RNA was extracted from the yielded supernatants in the same manner as that of Example 4. Using the extracted RNA as a sample (template), and using the same probes and the like as those of Example 1, 18S rRNA and GAPDH were amplified at the same time by the multiplex. The probe for 18S rRNA was labeled with FAM and the probe for GAPDH was labeled with VIC. The relative values of the signal intensities obtained from the assay are shown in FIG. 4. In the graph, the data of 18S rRNA are indicated by open circles, and the data of GAPDH are indicated by solid circles. The respective samples gave similar results as those of FIG. 1, showing that the same results can be obtained by the multiplex assay. Accordingly, this method is useful because the cancer detection accuracy is improved by detecting a plurality of markers at the same time, and thereby the cost and the sample quantity can be saved.

### Example 6

### (Methods)

Urine was collected from a healthy subject, 100 ml of which was divided into ten samples of 10 ml each in 50 ml tubes (manufactured by FALCON). Of these, five samples were freeze-dried and then preserved at 4° C. Of the remaining five samples, one sample was used as it was, and the remaining four samples were each added with 1 ml of the bladder cancer cell line EJ-1. These samples were mixed with a homogenizer to yield homogenized products. The homogenized samples were centrifuged at 4000×g for 10 minutes. Their supernatants were taken and RNA was extracted therefrom with the RNeasy kit (manufactured by QIAGEN). A portion of each extracted RNA, 21.5 μl of RNase-free water, 25 μl of 2× TaqMan Universal PCR Master Mix, and 2.5 μl of a primer/probe set respectively for CEA, GAPDH, and 18S rRNA (manufactured by Applied Biosystems) were put in a 0.2 ml PCR tube and mixed therein. The probe used here was a reporter probe having a fluorophore at one end and a quencher at the other end. These mixtures were subjected to nucleic acid amplification with a real-time fluorescence assay under a temperature condition consisting of one cycle of 95° C. for 20 seconds; and following 40 cycles of 95° C. for 3 seconds and 60° C. for 30 seconds, by using the 7500 Fast system (manufactured by Applied Biosystems). Plasmids containing cDNA of GAPDH, CEA, or 18S rRNA were used as standard substances for the copy number calculation, and were amplified at the same time.

### (Results)

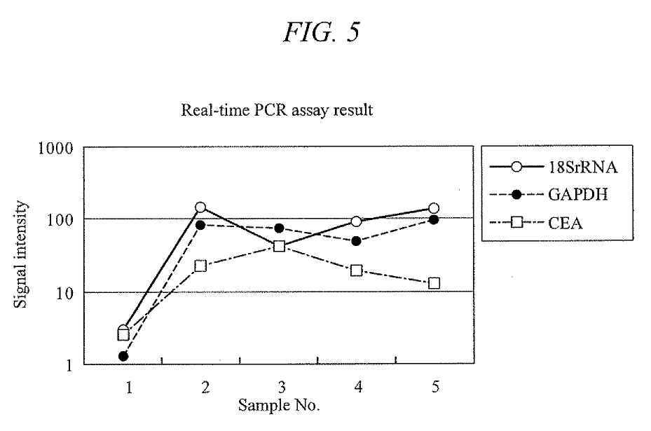

Signals from the nucleic acid amplification were obtained. FIG. 5 shows the summarized results of the fluorescence intensities from the patient samples and the healthy subject sample. In this graph, the sample No. 1 represents urine of the healthy subject and the sample Nos. 2 to 5 represent urine of the healthy subject including the bladder cancer cell line DJ-1. The data of 18S rRNA are indicated by open circles, the data of GAPDH are indicated by solid circles, and the data of CEA are indicated by open squares.

From about 20 ml of urine, all detection values of 18S rRNA, GAPDH, and CEA were small at under 5 in terms of the relative value in the healthy subject, while the detection values were over 10 in the other samples derived from the cancer cell line. Therefore, the threshold was able to be set at around 10 pg/μl per total RNA. These results showed that, if these housekeeping genes were used, it was possible to determine that values equal to or above the threshold of 10 pg/μl per total RNA were positive and values below this threshold were negative. By so doing, the presence of small quantities of normal cells which generated these housekeeping genes was found in the healthy subject, while the presence of large quantities of cancer cells which generated these housekeeping genes was proven in the cancer cell line. Moreover, the 18S rRNA and GAPDH values were higher than the CEA values, showing 18S rRNA and GAPDH had better performance than CEA.

As a result of the determination, the sample No. 1 had the 18S rRNA, GAPDH, and CEA values below the threshold of 10 and thus was determined to be normal, while the sample Nos. 2 to 5 had at least one of these values above the threshold of 10, which were twice or more than that of the normal sample No. 1, and thus were determined to be highly possibly cancerous.

### Example 7

Using the same samples as those of Example 6, 18S rRNA and GAPDH were amplified at the same time by the multiplex. At this time, the probe for 18S rRNA was labeled with FAM and the probe for GAPDH was labeled with VIC. As a result, the respective samples gave similar results as those of FIG. 2, showing that the same results can be obtained by the multiplex assay (not shown in a graph). Accordingly, this method is useful because the cancer detection accuracy is improved by detecting a plurality of markers at the same time, and thereby the cost and the sample quantity can be saved.

### Example 8

### (Methods)

100 ml of urine was each collected from six healthy subjects and six bladder cancer patients. These samples were centrifuged at 3000×g for 1 minute, and their residues were obtained. These residues were each added with 10 ml of PBS, and their precipitates were loosened. From the yielded products, the total protein was collected by a manual injector of the protein purification preparative system PLC-561i (manufactured by GL Sciences, 7810-15000), and subjected to electrophoresis using an SDS-polyacrylamide gel (manufactured by Bio-Rad). A nitrocellulose membrane was placed on the gel to transfer the proteins. Then, the membrane was stained with an anti-GAPDH antibody (manufactured by SIGMA, G9545) and then with an HRP-labeled secondary antibody. The +/−determination was made on the basis of the resultant data.

### (Results)

The results are shown in Table 1. The GAPDH protein was not detected from the healthy subjects (0/6=0%), whereas the GAPDH protein was detected from the bladder cancer patients (6/6=100%). These results showed that this housekeeping gene can be used in the bladder cancer test.

TABLE 1

| | Determination of anti-GAPDH antibody (+/−) |
|---|---|
| Healthy subject 1 | − |
| Healthy subject 2 | − |
| Healthy subject 3 | − |
| Healthy subject 4 | − |
| Healthy subject 5 | − |
| Healthy subject 6 | − |
| Bladder cancer patient 1 | + |
| Bladder cancer patient 2 | + |
| Bladder cancer patient 3 | + |
| Bladder cancer patient 4 | + |
| Bladder cancer patient 5 | + |
| Bladder cancer patient 6 | + |

### Example 9

RNA was respectively extracted from 0.5 to 1.0 g of feces from 75 colon cancer patients (five patients at stage 0, 13 patients at stage I, 28 patients at stage II, 16 patients at stage III, and 13 patients at stage IV), and 41 subjects of a control group, from which their cDNAs were produced. Their β2 microglobulin (B2M) expressions were quantified by real-time PCR (ABI7500 Fastsystem) with a standard sample whose copy number had been known, and compared with each other. The commercially available TaqMan (registered trademark) probe (manufactured by Applied Biosystems) was used as the B2M detection primer.

As a result, the median value of the B2M gene copy number was 6967 in the control group and 7639 in the colon cancer, which made no statistically significant difference (p=0.38, Mann-Whitney test). In the comparison between the control group and the stage III/IV, the median value was respectively 6967 and 29272, which made a significant difference of p=0.015. In the comparison within colon cancer patients between the stage 0/I/II and the stage III/IV, the stage III/IV was significantly superior regarding the B2M gene copy number (p=0.004). Moreover, in the comparison between occupation sites (left hemicolon and right hemicolon), no significant difference was found as the result was p=0.80.

In the early stage cancer at stage 0/I/II, cancer cells do exist but the number of exfoliated cells is small. This can be considered to be a reason why no difference was found in comparison with the control group. However, in the advanced cancer at stage III/IV, the number of exfoliated cells increases. This can be considered to be a reason why the significant difference was found.

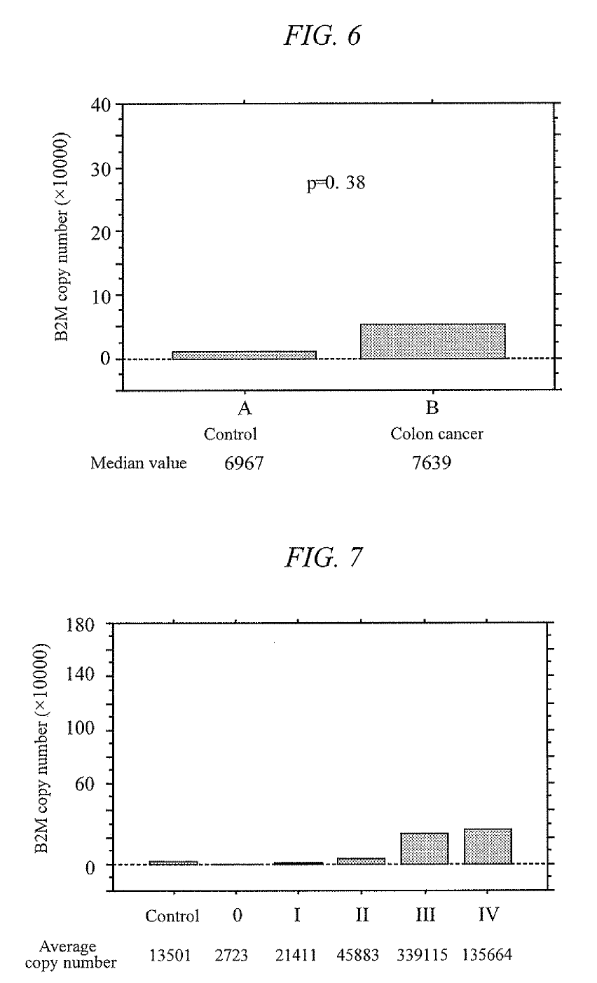

FIG. 6 shows the respective B2M gene copy numbers of the control group and the colon cancer patient group. FIG. 7 shows the respective B2M gene copy numbers of the control group and the colon cancer patient groups at respective stages. In the comparison between the results of the respective stages, the early stage cancer at stages 0/I/II showed almost equivalent B2M gene copy numbers to that of the control group, whereas the advanced cancer groups at stages III/IV showed notably greater scores B2M gene copy numbers. In particular, of the colon cancer patient groups, the stage III group showed the best score in the average B2M gene copy number.

From these results, it is apparent that advanced cancer such as stage III/IV colon cancer can be detected by measuring the B2M expression level (quantity of B2M-derived mRNA) in feces, that is, B2M can be used as a tumor marker per se for advanced cancer. In addition, B2M can be expected to provide highly reliable results regarding the stage of progression of cancer, if jointly used with another tumor marker. Therefore, B2M can also be used for the correction of tumor markers.

### Example 10

RNA was respectively extracted from 0.5 to 1.0 g of feces from 91 colon cancer patients and 45 subjects in a control group, from which their cDNAs were synthesized. Their COX2 and (β2 microglobulin (B2M) expressions were quantified by real-time PCR (ABI7500 Fastsystem) with a standard sample whose copy number had been known, and compared with each other. The commercially available TaqMan (registered trademark) probes (manufactured by Applied Biosystems) were used as the B2M detection primer and the COX2 detection primer.

On the basis of the COX2 copy number alone, the sensitivity was 85.7% (78/91) and the specificity was 93.3% (42/45). The detection values were corrected by using the values resulting from the division of the COX2 copy numbers by the B2M gene copy numbers (COX2 copy number/B2M gene copy number). On the basis of the correction values, the sensitivity was 94.5% (86/91) and the specificity was 95.6% (43/45). Significance tests of the sensitivity and the specificity were carried out between with and without the correction, by which a significant difference was found in the sensitivity (P=0.047), showing that the sensitivity was improved by performing the correction.

### Example 11

Feces was collected from a healthy subject, 9 g of which was placed in a 15 ml polypropylene tube (manufactured by FALCON) and was evenly mixed well. Then, the mixture was divided into two samples of 5 g and 4 g each. Of these, the sample of 5 g feces was added with 1 ml of a cell culture solution containing the colon cancer patient-derived cell line CCK-81 and 4 ml of PBS, and then well mixed. The resultant mixture was equally divided into five 15 ml polypropylene tubes at 1 ml each (samples A0 to A4). On the other hand, the sample of 4 g feces was added with 4 ml of PBS and then well mixed. The resultant mixture was equally divided into four 15 ml polypropylene tubes at 1 ml each (samples A5 to A8).

Of the five samples having the CCK-81 cell culture solution, one sample (sample A0) was used as a control of a directly sampled feces, which was immediately subjected to the nucleic acid recovery operation. Of the remaining samples, two samples (samples A1 and A2) were respectively added with 10 ml of ethanol (preparation solution for fecal samples) and mixed to effect immersion at normal temperature, before the nucleic acid recovery operation. The remaining two samples (samples A3 and A4) were preserved at 4° C. for 24 hours, before the nucleic acid recovery operation. In addition, of the four samples without the CCK-81 cell culture solution, two samples (samples A5 and A6) were respectively added with 10 ml of ethanol and mixed to effect immersion at normal temperature, before the nucleic acid recovery operation. The remaining two samples (samples A7 and A8) were preserved at 4° C. for 24 hours, before the nucleic acid recovery operation.

The nucleic acid recovery operation of each sample was performed as follows. First, each sample was respectively mixed and homogenized, from which impurities were removed by centrifugation. The resultant solution was added with 10 ml of an acid phenol-guanidine-chloroform solution, well mixed, and centrifuged at 4000×g for 10 minutes. The supernatant was portioned out. The supernatants thus prepared from the samples A0 to A8 were partially used for the measurement of the total RNA quantity in the supernatant with a UV spectrophotometer. As a result, the RNA concentration was between 190 ng and 510 ng/μl and the total recovered quantity of RNA was between 9.5 μg and 25.5 μg. The 260/230 nm absorbance ratio was respectively 1.8 or higher, which suggested that contamination with salts or the like was negligible. In addition, the 260/280 nm absorbance ratio was respectively 1.8 to 2.3, which suggested that contamination with proteins or the like was negligible. From these results of the RNA concentration, the total RNA quantity, and these two absorbance ratios (degree of purification), it was considered that the RNAs extracted/purified from the samples A0 to A8 had excellent quality and reliability. Therefore, the flow moved on to the next testing process.

In order to perform this RNA quality check from a different aspect, an assay was performed using the “Bioanalyzer” electrophoresis system of Agilent Technologies. As a result, bands of enterobacteria-derived 23Sand 16S ribosomal RNAs were found. The obtained RIN values were between 8.0 and 8.9, which were greater than 3 serving as the criterion for continuation of the test. Therefore, it was determined that the degree of fragmentation was low (the degree of decomposition was sufficiently low) and the quality was excellent. From this electrophoresis result, it was also considered that the RNAs extracted/purified from the samples A0 to A8 had excellent quality and reliability, and therefore, the flow was able to move on to the next testing process.

Appropriate amounts of these RNAs extracted/purified from the fecal samples were respectively taken, suitably diluted with TE, and normalized at 150 ng/μl.

These RNAs were subjected to RT-PCR by a usual method to obtain their cDNAs. 1 μl of each cDNA, 21.5 μl of RNase-free water, 25 μl of 2× TaqMan Universal PCR Master Mix, and 2.5 μl of a primer/probe set for the detection of the target gene (cyclooxygenase 2 (COX2) and glyceraldehyde 3-phosphate dehydrogenase (GAPDH)) (manufactured by Applied Biosystems) were put in a 0.2 ml PCR tube and mixed therein. Here, these probes were reporter probes labeled with a fluorophore at one end and a quencher at the other end.

These mixtures were treated at 95° C. for 2 minutes, and were subjected to nucleic acid amplification (PCR) with a real-time fluorescence assay under a reaction condition consisting of 40 cycles of 95° C. for 30 seconds and 60° C. for 1 minute, by using the 7900HT system (manufactured by Applied Biosystems). Plasmids containing cDNA of COX2 or GAPDH were used as control samples (standard substances) for the copy number calculation, and were amplified at the same time.

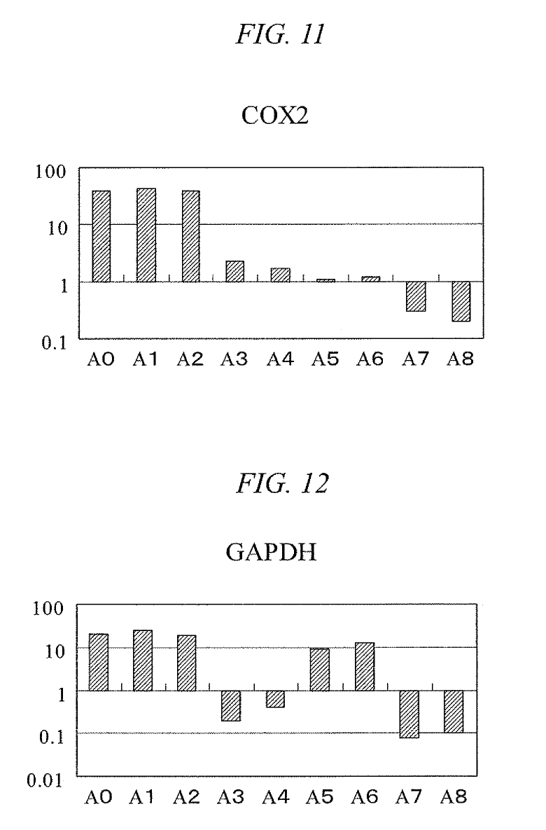

FIG. 11 and FIG. 12 show signals from nucleic acid amplification on respective samples, per each target gene. The y-axis in each graph is in a logarithmic scale on the basis of relative value. FIG. 11 shows the detection results of COX2 gene-derived nucleic acid (nucleic acid amplification product obtained from the template cDNA produced from the COX2 gene-derived RNA). On the other hand, FIG. 12 shows the detection results of GAPDH gene-derived nucleic acid (nucleic acid amplification product obtained from the template cDNA produced from the COX2 gene-derived RNA). As shown in FIG. 11, the quantity of COX2 was larger than the reference quantity (10) in the sample A0 which had been subjected to the nucleic acid extraction operation immediately after the fecal sampling and the samples A1 and A2 which had been added with and immersed in ethanol as the preparation solution for fecal samples. Therefore, the cancer cell-derived COX2 was able to be detected in these samples, and they were determined to be test positive.

On the other hand, as to two fecal samples (samples A5 and A6) without the CCK-81 cell culture solution, the quantity of the COX2 gene-derived nucleic acid was smaller than the reference quantity (10), and therefore these samples were determined to be test negative. In addition, as shown in FIG. 12, since the detected quantity of the nucleic acid derived from the GAPDH gene used as a standard gene having a constant expression level was larger than the reference value (1), it can be said that the testing process was successful and gave reliable data. In addition, as a different analysis method of the test result, a value resulting from the division of the quantity of the COX2 gene-derived nucleic acid by the quantity of the GAPDH gene-derived nucleic acid (COX2/GAPDH value) was respectively obtained. From this value, the COX2 gene expression level per unit of colon cells in feces can be obtained, which was found to be larger than the reference value (0.1).

In contrast, in the samples A3, A4, A7, and A8 which had not been added with and immersed in ethanol as the preparation solution for fecal samples, both the COX2 and the GAPDH were smaller than the detection reference values. Since the GAPDH quantity as a standard gene was smaller than the reference value, it can be considered that the RNAs extracted/purified from these samples were unreliable, and therefore the data (detection results) on the COX2 serving as the target gene was unreliable. In fact, in the samples A3 and A4 which had been added with the CCK-81 cell culture solution, the COX2 quantity was smaller than the reference value, and they were determined to be test negative. Thus, these determination results were apparently poor in reliability. In this manner, the qualities of the RNAs derived from the samples 1, 2, 5, and 6 were sufficient, whereas the qualities of the RNAs derived from the samples A3, A4, A7, and A8 were poor. This can be attributed to the difference between with and without the immersion in ethanol.

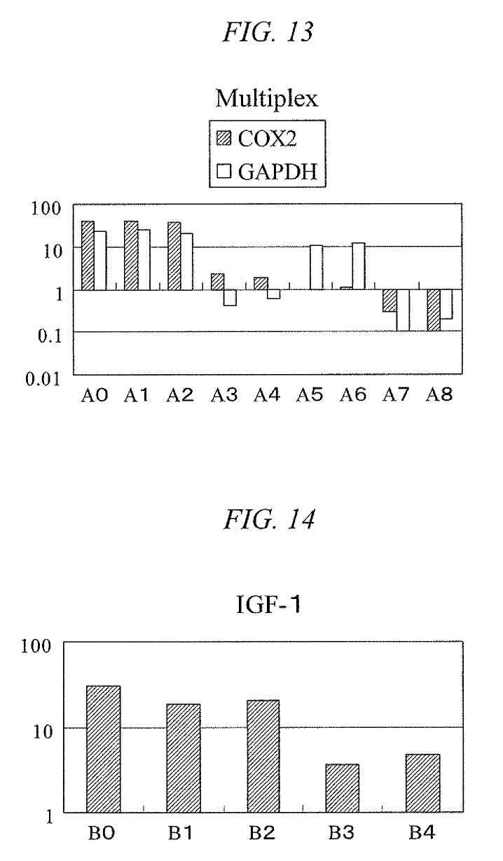

Furthermore, using the samples A0 to A2, A5, and A6, COX2 and GAPDH were amplified at the same time by the multiplex PCR. At this time, the probe for COX2 was labeled with FAM and the probe for GAPDH was labeled with VIC. FIG. 13 shows signals from the nucleic acid amplification on the respective samples, wherein the y-axis is in a logarithmic scale on the basis of relative value. The respective samples gave similar results as those of FIG. 11 and FIG. 12, showing that the assay was also possible by the multiplex PCR.

Moreover, in general, the quantity of cells contained in sampled feces depends on the condition of the feces. Here, the increase or decrease of the total nucleic acid quantity due to the condition of feces (correlated with the cell quantity) can be corrected by dividing the quantity of nucleic acid derived from the COX2 gene serving as the target gene (mRNA expression level of the COX2 gene) by the total nucleic acid quantity of feces (mRNA expression level of COX2 gene/total nucleic acid quantity of feces). The correction method by this calculation is effective particularly in cases where different fecal specimens are used (different examinees). However, in this example, well mixed and homogenized single feces was used, and thus the same effect as produced by the correction with the total nucleic acid quantity was achieved. Therefore, the correction was unnecessary.

### Example 12

Feces was collected from a healthy subject, 5 g of which was each placed in two 15 ml polypropylene tubes (manufactured by FALCON). The products were divided into samples of 3 g and 2 g each. Of these, the 3 g sample was added with 1 ml of a cell culture solution containing the colon cancer-derived cell line CCK-81 and 2 ml of PBS, and then well mixed. The resultant mixture was equally divided into three 15 ml polypropylene tubes (samples B0, B1, and B2). On the other hand, the 2 g sample was not added with the CCK-81 cell culture solution but added with 2 ml of PBS, and then well mixed. The resultant mixture was equally divided into two 15 ml polypropylene tubes (samples B3 and B4).

Of the three samples having the CCK-81 cells (samples B0 to B2), the sample B0 was immediately subjected to the extraction operation. The remaining samples B1 and B2 and other two samples without the CCK-81 cells (samples B3 and B4) were once frozen at −80° C., and then subjected to a centrifugal separation treatment to remove impurities. The resultant solutions were added with 10 ml of an acid phenol-guanidine-chloroform solution, well mixed, and centrifuged at 4000×g for 10 minutes. The supernatants were taken out therefrom, and subjected to the nucleic acid recovery operation. The nucleic acid recovery operation of these samples was performed in the same manner as that of Example 11.

The RNA-containing supernatants were partially used for the respective measurements of the RNA concentration and the total RNA quantity in supernatant, the 260/230 nm UV absorbance ratio, and the 260/280 nm UV absorbance ratio, with a UV spectrophotometer. As a result, in all cases, the concentration was 560 ng/μl or higher, exceeding the threshold (10). Moreover, the total RNA quantity was 29 μg for the sample B0, 35 μg for the sample B1, 28 μg for the sample B2, 32 μg for the sample B3, and 39 μg for the sample B4. On the other hand, in all cases, the 260/230 nm UV absorbance ratio was 2.0 or higher, and the 260/280 nm UV absorbance ratio was between 1.8 and 2.3.

From these results of the RNA concentration, the total RNA quantity, and these two absorbance ratios (degree of purification), it was considered that the RNAs extracted/purified from the samples B0 to B4 had excellent quality and reliability, and therefore, the flow moved on to the next testing process.

In order to normalize the quantities of the thus recovered RNAs of the respective samples at a constant concentration, appropriate amounts of RNAs were respectively taken, suitably diluted with TE, and normalized at 150 ng/μl. In addition, as an RNA control specimen, 10⁶ CCK-81 cells were charged in a 15 ml polypropylene tube (sample C1) and RNA was recovered therefrom using the RNeasy Mini Kit (manufactured by QIAGEN), then suitably diluted with TE, and normalized at 10 ng/μl.

These RNAs were subjected to RT-PCR by a usual method to obtain their cDNAs. 1 μl of each cDNA, 21.5 μl of RNase-free water, 25 μl of 2× TaqMan Universal PCR Master Mix, and 2.5 μl of a primer/probe set for the detection of the target gene (IGF-1 and β2 microglobulin (B2M)) (manufactured by Applied Biosystems) were put in a 0.2 ml PCR tube and mixed therein. Here, these probes were reporter probes labeled with a fluorophore at one end and a quencher at the other end.

These mixtures were treated at 95° C. for 2 minutes, and were subjected to nucleic acid amplification (PCR) with a real-time fluorescence assay under a reaction condition consisting of 40 cycles of 95° C. for 30 seconds and 60° C. for 1 minute, by using the 7900HT system (manufactured by Applied Biosystems). As a control sample (standard substance) for the copy number calculation, a plasmid was constructed by conjugating the pCR2.1 plasmid (manufactured by Invitrogen) with cDNA of the IGF-1 gene which had been isolated and extracted from the colon cancer (CCK-81) cell line, and a sample containing the plasmid (sample C2; concentration 1 ng/μl) was used. The plasmid held by the sample C2 was used as a control for forming a correction curve at the time of the nucleic acid amplification of the samples B0 to B4 by real-time PCR. The correction curve was formed from the results obtained by real-time PCR performed under the same condition but using a five-step dilution series (1-fold to 10000-fold) of the sample C2 as templates.

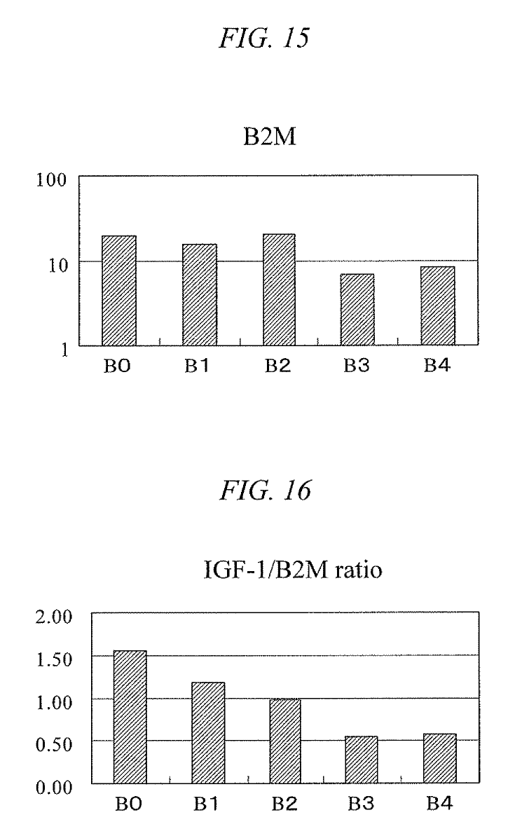

FIG. 14 shows the detection results of IGF-1 gene-derived nucleic acid (nucleic acid amplification product obtained from the template cDNA produced from the IGF-1 gene-derived RNA, hereunder, referred to as the IGF-1 expression level). FIG. 15 shows the detection results of B2M gene-derived nucleic acid (nucleic acid amplification product obtained from the template cDNA produced from the B2M gene-derived RNA, hereunder, referred to as the B2M expression level). Each graph shows the results of correction between samples with use of the results of the sample C2, regarding the signals from nucleic acid amplification on the respective samples. As a result, the samples B1 and B2 with the CCK-81 cells showed higher expression levels for both genes than the samples B3 and B4 without the CCK-81 cells. In particular, the samples B1 and B2 showed the IGF-1 expression level higher than the reference value of 10, and thus were determined to be test positive. In contrast, the samples B3 and B4 showed the IGF-1 expression level lower than the reference value of 10, and thus were determined to be test negative.

The determination was made on whether or not the series of the testing process was reliable, from the results of the RNA quantity, the RNA concentration, and the RNA quality, and/or the B2M expression level and the IGF-1 expression level, in accordance with the criteria of Table 2 or Table 3. In Table 2 and Table 3, the symbol “+” means the result in which the presence of the amplification product was found by PCR (expressed), and the symbol “−” means the result in which no presence of the amplification product was found by PCR (not expressed).

TABLE 2

| | (1) | (2) | (3) | (4) |
|---|---|---|---|---|
| Target gene | + | + | − | − |
| Standard gene | + | − | + | − |
| Reliability of | Reliable | Not reliable | Reliable | Not reliable |
| nucleic acid | | | | |
| quantity/quality | | | | |
| Determination: | Test | False positive | Test negative | False negative |
| test result | positive | | | |

TABLE 3

| | Nucleic acid quantity/quality | | | | | | | |
|---|---|---|---|---|---|---|---|---|
| | (1) | | | | (2) | | | |
| | RNA quantity/quality equal to or higher | | | | RNA quantity/quality | | | |
| | than threshold | | | | lower than threshold | | | |
| | (3) | (4) | (5) | (6) | (7) | (8) | (9) | (10) |
| Target gene | + | + | − | − | + | + | − | − |
| Standard gene | + | − | + | − | + | − | + | − |
| Reliability of | Yes | No | Yes | No | No | No | No | No |
| test result | | | | | | | | |

| | | | | | |
|---|---|---|---|---|---|
| Determination: | Positive | False | Negative | False | Suspected to be false |
| test result | | positive | | negative | positive or false negative |

As a result, in all samples, both the 260/230 nm UV absorbance ratio and the 260/280 nm UV absorbance ratio were within the reference range, and thus the RNA quality was determined to be excellent. The recovered RNA quantity was over 1 μg in all samples B0 to B4, and thus the testing process was determined to be excellent. Furthermore, the IGF-1 and B2M expressions were detected (>0) in all samples, and thus the testing process was determined to be satisfactorily performed. From these results, the process was confirmed to be reliable.

Moreover, as an option, the expression level of IGF-1 serving as the target gene was corrected by dividing it by the expression level of B2M used as the standard gene having a constant expression level (IGF-1 expression level/B2M expression level) to obtain the expression level per cell, with which the comparison was made. FIG. 16 shows the results of the correction. In these results, the expression levels of the samples B1 and B2 were clearly different from those of the samples B3 to B4. That is, it was clarified that the sensitivity and the specificity of the adenoma or cancer test can be further improved by dividing the quantity of the target gene-derived RNA by the quantity of the standard gene-derived RNA.

### INDUSTRIAL APPLICABILITY

The present invention enables an early stage detection of cancer by genetic analysis of a biomarker in a readily collectable sample. Moreover, the testing process of a nucleic acid in feces can be performed with higher reliability by using the adenoma or cancer testing method of the present invention. Since a target nucleic acid accounting for a very small population in feces can be measured and analyzed with high accuracy, the present invention can be utilized in the fields of clinical tests or the like which use fecal samples, in particular, in the field of adenoma or cancer diagnosis which is required to provide highly reliable diagnosis results.

1. (canceled)

2. An adenoma or cancer detection method comprising:
(i) extracting a nucleic acid from a sample of feces or urine that has been collected from an examinee; and
(ii) measuring the quantity of an expression product of a sequence constituting at least one housekeeping gene in the extracted nucleic acid, and calculating the concentration of the sequence in the sample.

3. The adenoma or cancer detection method according to claim 2, wherein the method further comprises:
(a) freezing or freeze-drying it at 0° C. or lower temperature, or treating it with alcohol or an alcoholic solution, and then homogenizing the sample before (i).

4. The adenoma or cancer detection method according to claim 2, further comprising:
(b) producing a cDNA from an RNA extracted in (i), which is derived from at least one housekeeping gene, between (i) and (ii); and
(ii-1) quantifying the cDNA in (ii).

5. (canceled)

6. The adenoma or cancer detection method according to claim 2, wherein there are two or more types of the housekeeping genes, and a plurality of quantities of sequences constituting these housekeeping genes or expression products thereof are measured at the same time.

7. The adenoma or cancer detection method according to claim 6, wherein the housekeeping gene is a gene selected from the group consisting of glyceraldehyde-3-phosphate dehydrogenase (GAPDH), 18S ribosomal RNA, 28S ribosomal RNA, β actin, β2 microglobulin, hypoxanthine phosphoribosyl transferase 1, ribosomal protein large P0, peptidylpropyl isomerase A (cyclosporin A), cytochrome C, phosphoglycerate kinase 1, β-glucuronidase, TATA box binding factor, transferrin receptor, HLA-A0201 heavy chain, ribosomal protein L19, α tubulin, β tubulin, γ tubulin, ATP synthetase, eukaryotic translation elongation factor 1 gamma (EEF1G), succinate dehydrogenase complex (SDHA), aminolevulinic acid synthase 1 (ALAS1), ADP-ribosylation factor 6, endonuclease G (ENDOG), and peroxisomal biogenesis factor (PEX).

8. The adenoma or cancer detection method according to claim 2, wherein the excrement sample is feces and the housekeeping gene is selected from the group consisting of: β2 microglobulin, glyceraldehydes-3-phosphate dehydrogenase (GAPDH), 18S ribosome RNA, and b actin.

9. The adenoma or cancer detection method according to claim 2 wherein the method further comprises, after (ii):
(iii) measuring the quantity of an expression product of a sequence constituting at least one tumor gene in the extracted nucleic acid, and calculating the concentration of the sequence in the sample; and
(iv) correcting the concentration of the expression product of the sequence constituting the tumor gene that has been calculated in (iii), based on the concentration of the expression product of the sequence constituting the housekeeping gene that has been calculated in (ii).

10. An adenoma or cancer testing method for testing an adenoma or cancer with use of a marker gene for the adenoma or cancer (the target gene), which comprises:
(A) extracting an RNA contained in feces that has been collected from an examinee, and purifying it as an RNA solution;
(B) measuring the quantity of a target gene-derived RNA in the RNA solution obtained in the step (A);
(C) comparing the quantity of the target gene-derived RNA obtained in (B) with a preset threshold, to determine whether or not the examinee is affected by the adenoma or cancer;
(D) measuring one or more items selected from the group consisting of the degree of RNA purification, the degree of RNA decomposition, the RNA concentration, and the quantity of a standard gene-derived RNA, in the RNA solution obtained in (A);
(E) judging the reliability of the RNA in the RNA solution obtained in (A), based on the value obtained in (D); and
(F) judging that the determination of (C) is reliable if the RNA is judged to be reliable in (E), and judging that the determination of (C) is unreliable if the RNA is judged to be unreliable in (E);
provided that (B), (C), (D), and (E) may be carried out in the order of (D), (E), (B), and (C); (B), (D), (C), and (E); (B), (D), (E), and (C); (D), (B), (E), and (C); or (D), (B), (C), and (E).

11. The adenoma or cancer testing method according to claim 10, wherein (B), (C), (D), and (E) are carried out in the order of (D), (E), (B), and (C); and (B) is the following (B1), and (C) is the following (C1):
(B1) terminating the test if the RNA is judged to be unreliable in (E), or measuring the quantity of a target gene-derived RNA in the RNA solution obtained in (A) if the RNA is judged to be reliable in (E); and
(C1) comparing the quantity of the target gene-derived RNA obtained in (B1) with a preset threshold, to determine whether or not the examinee is affected by the adenoma or cancer.

12. The adenoma or cancer testing method according to claim 10, wherein (B), (C), (D), and (E) are carried out in the order of (B), (D), (E), and (C); and (D) is the following (D2), (E) is the following (E2), and (C) is the following (C2):
(D2) measuring one or more items selected from the group consisting of the degree of RNA purification, the degree of RNA decomposition, the RNA concentration, and the quantity of a standard gene-derived RNA, in the RNA solution obtained in (A), after (B);
(E2) judging the reliability of the RNA in the RNA solution obtained in (A), based on the value obtained in (D2); and
(C2) terminating the test if the RNA is judged to be unreliable in (E2), or comparing the quantity of the target gene-derived RNA obtained in (B) with a preset threshold, to determine whether or not the examinee is affected by the adenoma or cancer, if the RNA is judged to be reliable in (E2).

13. (canceled)

14. The adenoma or cancer testing method according to claim 10, wherein the measurement of the degree of RNA purification is carried out by measuring a value resulting from the division of the absorbance at 260 nm by the absorbance at 230 nm (260/230 nm absorbance ratio) of the RNA solution obtained in (A), and/or a value resulting from the division of the absorbance at 260 nm by the absorbance at 280 nm (260/280 nm absorbance ratio) thereof.

15-18. (canceled)

19. The adenoma or cancer testing method according to claim 10, wherein the RNA obtained in (A) is judged to be unreliable if the quantity of the standard gene-derived RNA is smaller than a preset threshold.

20. The adenoma or cancer testing method according to claim 10, wherein the measurement of the quantity of the target gene-derived RNA is carried out after normalization of the RNA solution obtained in (A).

21. The adenoma or cancer testing method according to claim 10, wherein the measurement of the quantity of the target gene-derived RNA is carried out by nucleic acid amplification with use of a cDNA, as a template, which has been obtained from a reverse transcription reaction of the RNA in the RNA solution obtained in (A).

22. The adenoma or cancer testing method according to claim 10, wherein the measurement of the quantity of the target gene-derived RNA is carried out by performing a reverse transcription reaction after normalization of the RNA solution obtained in the step (A), and nucleic acid amplification with use of a resulting cDNA as a template.

23. (canceled)

24. The adenoma or cancer testing method according to claim 10, wherein the standard gene is a housekeeping gene or an epithelial cell-specific gene.

25. The adenoma or cancer testing method according to claim 24, wherein the epithelial cell-specific gene is a gene selected from the group consisting of a carcinoembryonic antigen gene, a cell adhesion factor gene, a mucin gene, and a cytokeratin gene.

26. The adenoma or cancer testing method according to claim 10, wherein the target gene is a gene selected from the group consisting of cyclooxygenase 2 (COX2), matrix metallopeptidase 7 (MMP7), and SNAIL.

27. The adenoma or cancer testing method according to claim 24, wherein the standard gene is a gene selected from the group consisting of glyceraldehyde-3-phosphate dehydrogenase (GAPDH), 18S ribosomal RNA, 28S ribosomal RNA, β actin, β2 microglobulin, hypoxanthine phosphoribosyl transferase 1, ribosomal protein large P0, peptidylpropyl isomerase A (cyclosporin A), cytochrome C, phosphoglycerate kinase 1, β-glucuronidase, TATA box binding factor, transferrin receptor, HLA-A0201 heavy chain, ribosomal protein L19, α tubulin, β tubulin, γ tubulin, ATP synthetase, eukaryotic translation elongation factor 1 gamma (EEF1G), succinate dehydrogenase complex (SDHA), aminolevulinic acid synthase 1 (ALAS1), ADP-ribosylation factor 6, endonuclease G (ENDOG), peroxisomal biogenesis factor (PEX), carcinoembryonic antigen (CEA), epithelial cell adhesion molecule (EpCAM), mutin 2 (MUC2), mutin 3 (MUC3), mutin 4 (MUC4), keratin 7 (CK7), keratin 19 (CK19), and keratin 20 (CK20).

28. The adenoma or cancer detection method according to claim 2, wherein the adenoma or the cancer is colon adenoma, colon cancer, or bladder cancer.

29. A colon adenoma detection method, comprising:
(i) freezing or freeze-drying a feces sample collected from an examinee, or treating the sample with alcohol or an alcoholic solution at 0° C. or below, and then homogenizing the sample;
(ii) extracting RNA from the feces sample treated in the step (i);
(iii) producing cDNAs from an RNA derived from a b2 microglobulin gene and an RNA derived from at least one tumor gene, both being contained in the RNA extracted in (ii);
(iv) quantifying the cDNAs obtained in (iii), and calculating copy numbers of the RNA derived from the b2 microglobulin gene and the RNA derived from the tumor gene;
(v) correcting the copy number of the RNA derived from the tumor gene calculated in (iv), based on the copy number of the RNA derived from the b2 microglobulin gene calculated in (iv).

30. A method for determining a stage of a colon adenoma or a colon cancer comprising:
(i) extracting an RNA from a feces sample collected from an examinee;
(ii) producing a cDNA from an RNA derived from a b2 microglobulin gene contained in the RNA extracted in (i);
(iii) quantifying the cDNA of (ii), and calculating a copy number of the RNA derived from the b2 microglobulin gene;
(iv) comparing the copy number of the b2 microglobulin gene calculated in (iii) with the copy number of the RNA in a control sample.

31. The method for determining a stage of a colon adenoma or a colon cancer according to claim 30, further comprising
(v) determining the colon adenoma of the colon cancer is in Stage III/IV, if the copy number of the RNA derived from the b2 microglobulin gene is significantly larger than the copy number of the RNA in the control sample in the comparison result in (iv).

32-33. (canceled)
